SEQUENCE LISTING (27 sequences)

SEQ ID NO: 1 - DNA, 27 bp, Artificial Sequence
  Primer
gtgctaggtaactaacgtttgattttt

SEQ ID NO: 2 - DNA, 20 bp, Artificial Sequence
  Primer
aaccttccacgttagcttga

SEQ ID NO: 3 - DNA, 20 bp, Artificial Sequence
  Primer
gcattagttggaccaccttg

SEQ ID NO: 4 - DNA, 20 bp, Artificial Sequence
  Primer
atcacctgaagcaggtttga

SEQ ID NO: 5 - DNA, 23 bp, Artificial Sequence
  Primer
gcactacctgatgaaaaataacc

SEQ ID NO: 6 - DNA, 20 bp, Artificial Sequence
  Primer
ccgaactgaggttgggttta

SEQ ID NO: 7 - DNA, 20 bp, Artificial Sequence
  Primer
gggggagcgaataggattag

SEQ ID NO: 8 - DNA, 23 bp, Artificial Sequence
  Primer
ggaaggggacgtaggtacataaa

SEQ ID NO: 9 - DNA, 23 bp, Artificial Sequence
  Primer
ttagaacgtgttttgttcccaat

SEQ ID NO: 10 - DNA, 24 bp, Artificial Sequence
  Primer
tggtacaagaggatttttgttgtt

SEQ ID NO: 11 - DNA, 23 bp, Artificial Sequence
  Primer
aaatttaacgtaacgatgagttg

SEQ ID NO: 12 - DNA, 20 bp, Artificial Sequence
  Primer
ccccttacgggcaagtaaac

SEQ ID NO: 13 - DNA, 20 bp, Artificial Sequence
  Primer
ctcgcctatcggctaacaag

SEQ ID NO: 14 - DNA, 20 bp, Artificial Sequence
  Primer
cacaagaagcaaccccttga

SEQ ID NO: 15 - DNA, 10026 bp, Artificial Sequence
  Codon optimized sequence
gcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagcactagtggatcgcccgggctgcaggaattccatatttagataaacgatttcaagcagcagaattagctttattagaacaaacttgtaaagaaatgaatgtaccaatgccgcgcattgtagaaaaaccagataattattatcaaattcgacgtatacgtgaattaaaacctgatttaacgattactggaatggcacatgcaaatccattagaagctcgaggtattacaacaaaatggtcagttgaatttacttttgctcaaattcatggatttactaatacacgtgaaattttagaattagtaacacagcctcttagacgcaatctaatgtcaaatcaatctgtaaatgctatttcttaatataaatcccaaaagattttttttataatactgagacttcaacacttacttgtttttattttttgtagttacaattcactcacgttaaagacattggaaaatgaggcaggacgttagtcgatatttatacactcttaagtttacttgcccaatatttatattaggacgtccccttcgggtaaataaattttagtggcagtggtaccaccactgcctattttaatactccgaagcatataaatatacttcggagtatataaatatccactaatatttatattaggcagttggcaggcaacaataaataaatttgtcccgtaaggggacgtcccgaaggggaaggggaagaaggcagttgcctcgcctatcggctaacaagttcctttggagtatataaccgcctacaggtaacttaaagaacatttgttacccgtaggggtttatacttctaattgcttcttctgaacaataaaatggtttgtgtggtctgggctaggaaacttgtaacaatgtgtagtgtcgcttccgcttcccttcgggacgtccccttcgggtaagtaaacttaggagtattaaatcgggacgtccccttcgggtaaataaatttcagtggacgtccccttacgggacgccagtagacgtcagtggcagttgcctcgcctatcggctaacaagttccttcggagtatataaatatagaatgtttacatactcctaagtttacttgcctccttcggagtatataaatatcccgaaggggaaggaggacgccagtggcagtggtaccgccactgcctgcttcctccttcggagtatgtaaaccccttcgggcaactaaagtttatcgcagtatataaatataggcagttggcaggcaactgccactgacgtcctattttaatactccgaaggaggcagttggcaggcaactgccactgacgtcccgtaagggtaaggggacgtccactggcgtcccgtaaggggaaggggacgtaggtacataaatgtgctaggtaactaacgtttgattttttgtggtataatatatgtaccatgcttttaatagaagcttgaatttataaattaaaatatttttacaatattttacggagaaattaaaactttaaaaaaattaacatatggtaccaaacaaaagcgtagcaccattattacttgctgcatctatcttatatggtggtgctgttgctcaacagactgtttggggtcagtgtggtggtattggttggtctggtcctaccaattgtgctcctggctcagcatgtagtaccttaaatccttactatgctcaatgtattccaggtgcaacaactataacaacatcaactcgccctccttcaggtccaactacaacaactcgtgctactagcacttctagcagcacacctcctacatcttctggagtacgtttcgctggtgttaatattgcaggtttcgattttggttgtactaccgatggtacatgtgttaccagtaaagtttatccccctttaaaaaattttactggctcaaacaattatccagatggcattggtcaaatgcaacactttgtaaatgaagatggtatgactattttccgtttaccagtgggctggcaatacttagttaacaacaatttaggtggtaacttagatagtacatcaattagtaaatatgatcaattagtacaaggttgcttatctttaggtgcctattgtattgttgatattcataattatgcccgttggaacggtggtattattggtcaaggtggtccaactaatgctcaatttacatcattatggagccaattagcttcaaaatatgctagtcaatcacgtgtttggttcggtattatgaatgaacctcacgatgtgaacataaatacttgggctgcaactgtgcaagaagtagtaactgctattcgtaatgctggtgcaacatcacaattcattagtttaccaggcaacgattggcaatctgccggcgcttttatttctgacggtagcgcagctgctcttagtcaagtgactaacccagacggtagtaccactaacttaatattcgatgtacataaatatcttgattctgataatagcggaacacacgccgaatgtaccacaaataatattgatggtgcttttagtcctttagcaacttggttacgtcaaaataatcgccaagccattttaactgaaacaggtggtggaaacgtgcagagttgtatccaagacatgtgtcaacaaattcagtacttaaatcaaaactctgacgtgtacttaggttatgtaggttggggtgctggttcttttgattcaacttatgtattaaccgaaacccctacttcttctggaaactcatggacagacacttcattagtaagtagttgtttagctcgcaagggtaccggtgaaaacttatactttcaaggctcaggtggcggtggaagtgattacaaagatgatgatgataaaggaaccggttaatctagacttagcttcaactaactctagctcaaacaactaatttttttttaaactaaaataaatctggttaaccatacctggtttattttagtttagtttatacacacttttcatatatatatacttaatagctaccataggcagttggcaggacgtccccttacgggacaaatgtatttattgttgcctgccaactgcctaatataaatattagtggacgtccccttccccttacgggcaagtaaacttagggattttaatgctccgttaggaggcaaataaattttagtggcagttgcctcgcctatcggctaacaagttccttcggagtatataaatatcctgccaactgccgatatttatatactaggcagtggcggtaccactcgacggatcctacgtaatcgatgaattcgatcccatttttataactggtctcaaaatacctataaacccattgttcttctcttttagctctaagaacaatcaatttataaatatatttattattatgctataatataaatactatataaatacatttacctttttataaatacatttaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttcttttttcgtatggaattgcccaatattattcaacaatttatcggaaacagcgttttagagccaaataaaattggtcagtcgccatcggatgtttattcttttaatcgaaataatgaaactttttttcttaagcgatctagcactttatatacagagaccacatacagtgtctctcgtgaagcgaaaatgttgagttggctctctgagaaattaaaggtgcctgaactcatcatgacttttcaggatgagcagtttgaatttatgatcactaaagcgatcaatgcaaaaccaatttcagcgctttttttaacagaccaagaattgcttgctatctataaggaggcactcaatctgttaaattcaattgctattattgattgtccatttatttcaaacattgatcatcggttaaaagagtcaaaattttttattgataaccaactccttgacgatatagatcaagatgattttgacactgaattatggggagaccataaaacttacctaagtctatggaatgagttaaccgagactcgtgttgaagaaagattggttttttctcatggcgatatcacggatagtaatatttttatagataaattcaatgaaatttattttttagaccttggtcgtgctgggttagcagatgaatttgtagatatatcctttgttgaacgttgcctaagagaggatgcatcggaggaaactgcgaaaatatttttaaagcatttaaaaaatgatagacctgacaaaaggaattattttttaaaacttgatgaattgaattgattccaagcattatctaaaatactctgcaggcacgctagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaatttttatttttcatgatgtttatgtgaatagcataaacatcgtttttattttttatggtgtttaggttaaatacctaaacatcattttacatttttaaaattaagttctaaagttatcttttgtttaaatttgcctgtgctttataaattacgatgtgccagaaaaataaaatcttagctttttattatagaatttatctttatgtattatattttataagttataataaaagaaatagtaacatactaaagcggatgtagcgcgtttatcttaacggaaggaattcggcgcctacgtaggatccgtatccatgctagcaatatctgatggtacttgcatttcataagtttggcctggaataaccaccgtttcggaagtacctgtcgctttaagttttatagctaaatctaaagtttctttaagtcttttagctgtattaaatactccacgactttcccttacgggacaataaataaatttgtccccttccccttacgtgacgtcagtggcagttgcctgccaactgcctccttcggagtattaaaatcctatatttatatactcctaagtttacttgcccaatatttatattaggcagttggcaggcaactgccactgacgtcccgaaggggaaggggaaggacgtccccttcgggtaaataaattttagtggcagtggtaccaccactgcctgcttcctccttccccttcgggcaagtaaacttagaataaaatttatttgctgcgctagcaggtttacatactcctaagtttacttgcccgaaggggaaggaggacgtccccttacgggaatataaatattagtggcagtggtacaataaataaattgtatgtaaaccccttcgggcaactaaagtttatcgcagtatataaatatagaatgtttacatactccgaaggaggacgccagtggcagtggtaccgccactgcctgtccgcagtattaacatcctattttaatactccgaaggaggcagttggcaggcaactgccactaatatttatattcccgtaaggggacgtcctaatttaatactccgaaggaggcagttggcaggcaactgccactaaaatttatttgcctcctaacggagcattaaaatcccgaaggggacgtcccgaaggggaaggggaaggaggcaactgcctgcttcctccttccccttcgggcaagtaaacttagaataaaatttatttgctgcgctagcaggtttacatactcctaagtttacttgcccgaaggggaaggaggacgtccccttacgggaatataaatattagtggcagtggtacaataaataaattgtatgtaaaccccttcgggcaactaaagtttatcgcagtatataaatatcggcagttggcaggcaactgccactaaaattcatttgcccgaaggggacgtccactaatatttatattcccgtaaggggacgtcccgaaggggaaggggacgtcctaaacggagcattaaaatccctaagtttacttgcctaggcagttggcaggatatttatatacgatattaatacttttgctactggcacactaaaatttatttgcccgtaaggggacgtccttcggtggttatataaataatcccgtagggggagggggatgtcccgtagggggaggggagtggaggctccaacggaggttggagcttctttggtttcctaggcattatttaaatattttttaaccctagcactagaactgagattccagacggcgacccgtaaagttcttcagtcccctcagctttttcacaaccaagttcgggatggattggtgtgggtccaactgagcaaagagcaccaaggttaactgcatctctgtgagatgctagttaaactaagcttagcttagctcataaacgatagttacccgcaaggggttatgtaattatattataaggtcaaaatcaaacggcctttagtatatctcggctaaagccattgctgactgtacacctgatacctatataacggcttgtctagccgcggccttagagagcactcatcttgagtttagcttcctacttagatgctttcagcagttatctatccatgcgtagctacccagcgtttcccattggaatgagaactggtacacaattggcatgtcctttcaggtcctctcgtactatgaaaggctactctcaatgctctaacgcctacaccggatatggaccaaactgtctcacgcatgaaattttaaagccgaataaaacttgcggtctttaaaactaacccctttactttcgtaaaggcatggactatgtcttcatcctgctactgttaatggcaggagtcggcgtattatactttcccactctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtg

SEQ ID NO: 16 - protein, 447 aa, Artificial Sequence
  Codon optimized sequence
MVPNKSVAPLLLAASILYGGAVAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNEDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTSSGNSWTDTSLVSSCLARKGTGENLYFQGSGGGGSDYKDDDDKGTG

SEQ ID NO: 17 - DNA, 1191 bp, Artificial Sequence
  Codon optimized sequence
atggtaccacacaagttcacaggtgttaacgctaaattccagcaaccagcattaagaaatttatctccagtggtagttgagcgcgaacgtgaggaatttgtaggattctttccacaaattgttcgtgacttaactgaagatggtattggtcatccagaagtaggtgacgctgtagctcgtcttaaagaagtattacaatacaacgcacctggtggtaaatgcaatagaggtttaacagttgttgcagcttaccgtgaactttctggaccaggtcaaaaagacgctgaaagtcttcgttgtgctttagcagtaggatggtgtattgaattattccaagcctttttcttagttgctgacgatataatggaccagtcattaactagacgtggtcaattatgttggtacaagaaagaaggtgttggtttagatgcaataaatgattcttttcttttagaaagctctgtgtatcgcgttcttaaaaagtattgccgtcaacgtccatattatgtacatttattagagctttttcttcaaacagcttaccaaacagaattaggacaaatgttagatttaatcactgctcctgtatctaaggtagatttaagccatttctcagaagaacgttacaaagctattgttaagtataaaactgctttctattcattctatttaccagttgcagcagctatgtatatggttggtatagattctaaagaagaacatgaaaacgcaaaagctattttacttgagatgggtgaatacttccaaattcaagatgattatttagattgttttggcgatcctgctttaacaggtaaagtaggtactgatattcaagataacaaatgttcatggttagttgtgcaatgcttacaaagagtaacaccagaacaacgtcaacttttagaagataattacggtcgtaaagaaccagaaaaagttgctaaagttaaagaattatatgaggctgtaggtatgagagccgcctttcaacaatacgaagaaagtagttaccgtcgtcttcaagagttaattgagaaacattctaatcgtttaccaaaagaaattttcttaggtttagctcagaaaatatacaaacgtcaaaaaggtaccggtgaaaacttatactttcaaggctcaggtggcggtggaagtgattacaaagatgatgatgataaaggaaccggttaa

SEQ ID NO: 18 - DNA, 2511 bp, Artificial Sequence
  Codon optimized sequence
atggtaccaacaagtgtatcagtagaatcaggaacagtatcttgtttatcatcaaacaacttaattagacgtacagctaatccacatcctaacatttggggatatgattttgttcactcacttaaatcaccatatacacacgactcatcatatcgtgaacgtgctgagactttaatttcagaaataaaagttatgcttggaggtggtgaattaatgatgactccatcagcttatgatacagcatgggtagctcgtgttccatcaattgacggtagtgcttgtccacaatttccacaaactgttgaatggattcttaaaaaccaattaaaagatggtagttggggaactgaatctcacttcttacttagtgacagattattagctacattaagttgtgtattagcattattaaaatggaaagtagctgatgttcaagtagagcaaggtattgagtttatcaaacgtaatttacaagctattaaagacgaacgtgatcaagacagtttagtaactgatttcgagattattttcccatcacttttaaaagaggctcaatctttaaacttaggcttaccttatgatttaccatatattagattattacaaacaaaacgtcaagaacgtcttgctaacttaagtatggataaaattcacggtggtactttattatcatctttagagggcattcaagatatagttgaatgggaaacaattatggatgtacaatctcaagatggttctttcttatcatcaccagcttctacagcatgtgtattcatgcatacaggagatatgaaatgtttagatttcttaaacaacgtattaactaaatttggtagtagtgttccttgtttataccctgtagatttattagaacgtcttttaattgtagataatgtagagcgtcttggtattgaccgtcattttgaaaaagaaatcaaagaggctttagattatgtttatcgtcattggaacgatcgtggtattggttggggtcgtttatcacctatcgcagacttagaaacaacagctttaggttttcgtttacttcgtcttcatcgttacaatgtttctcctgtagtattagacaatttcaaagacgcagatggcgagttcttctgcagtacaggtcaatttaacaaagatgttgcaagtatgttatctttataccgtgcttctcaattagctttccctgaagaatcaattttagatgaagctaaatcattctcaacacaatatcttcgtgaagcattagaaaaatcagaaacattttcttcttggaatcatcgtcagagtttatcagaagaaattaaatatgctttaaaaacatcatggcacgcttcagttcctcgtgttgaagcaaaacgttattgtcaggtttaccgtcaagactatgctcatttagcaaaatcagtttataaacttcctaaagtaaataatgagaaaattcttgaattagcaaaattagattttaacattattcaatctatccatcaaaaagaaatgaaaaatgttacatcatggtttcgtgattcaggcttaccacttttcacatttgctcgtgaaagacctttagagttttactttttaatcgctggtggaacatacgaacctcaatacgcaaaatgtagattcttatttacaaaagtagcttgtttacaaactgttttagacgatatgtacgatacttacggtacaccatcagagttaaaattatttactgaggcagttcgtcgttgggatttatcattcacagaaaacttacctgattatatgaaattatgctacaaaatttactatgatattgttcatgaagttgcttgggaagtagaaaaagaacagggacgtgagcttgtttcatttttccgtaaaggttgggaagactatcttttaggttattatgaagaagctgaatggttagctgctgaatacgttcctactttagatgaatacattaaaaacggtattacatctattggtcaacgtattttacttttatcaggtgtacttattatggaaggtcaacttttatcacaagaagctcttgaaaaagtagattatccaggtcgtcgtgttttaacagaattaaacagtttaattagtcgtttagcagacgatactaaaacatacaaagcagaaaaagctcgtggtgaacttgctagtagtattgaatgttatatgaaagaccaccctggttgtcaagaagaagaagcattaaaccatatttatggcattttagaaccagctgttaaagaattaactcgtgagtttcttaaagcagatcacgtaccattcccttgcaaaaaaatgttatttgatgaaacaagagttacaatggtaattttcaaagatggtgatggtttcggtatttctaaattagaagtaaaagaccacataaaagaatgtttaattgagccattaccacttggtaccggtgaaaatctttattttcaaggtagtggtggtggcggttctgactacaaagatgacgacgataaaggaaccggttaa

SEQ ID NO: 19 - DNA, 2526 bp, Artificial Sequence
  Codon optimized sequence
catatggtaccaacaagtgtatcagtagaatcaggaacagtatcttgtttatcatcaaacaacttaattagacgtacagctaatccacatcctaacatttggggatatgattttgttcactcacttaaatcaccatatacacacgactcatcatatcgtgaacgtgctgagactttaatttcagaaataaaagttatgcttggaggtggtgaattaatgatgactccatcagcttatgatacagcatgggtagctcgtgttccatcaattgacggtagtgcttgtccacaatttccacaaactgttgaatggattcttaaaaaccaattaaaagatggtagttggggaactgaatctcacttcttacttagtgacagattattagctacattaagttgtgtattagcattattaaaatggaaagtagctgatgttcaagtagagcaaggtattgagtttatcaaacgtaatttacaagctattaaagacgaacgtgatcaagacagtttagtaactgatttcgagattattttcccatcacttttaaaagaggctcaatctttaaacttaggcttaccttatgatttaccatatattagattattacaaacaaaacgtcaagaacgtcttgctaacttaagtatggataaaattcacggtggtactttattatcatctttagagggcattcaagatatagttgaatgggaaacaattatggatgtacaatctcaagatggttctttcttatcatcaccagcttctacagcatgtgtattcatgcatacaggagatatgaaatgtttagatttcttaaacaacgtattaactaaatttggtagtagtgttccttgtttataccctgtagatttattagaacgtcttttaattgtagataatgtagagcgtcttggtattgaccgtcattttgaaaaagaaatcaaagaggctttagattatgtttatcgtcattggaacgatcgtggtattggttggggtcgtttatcacctatcgcagacttagaaacaacagctttaggttttcgtttacttcgtcttcatcgttacaatgtttctcctgtagtattagacaatttcaaagacgcagatggcgagttcttctgcagtacaggtcaatttaacaaagatgttgcaagtatgttatctttataccgtgcttctcaattagctttccctgaagaatcaattttagatgaagctaaatcattctcaacacaatatcttcgtgaagcattagaaaaatcagaaacattttcttcttggaatcatcgtcagagtttatcagaagaaattaaatatgctttaaaaacatcatggcacgcttcagttcctcgtgttgaagcaaaacgttattgtcaggtttaccgtcaagactatgctcatttagcaaaatcagtttataaacttcctaaagtaaataatgagaaaattcttgaattagcaaaattagattttaacattattcaatctatccatcaaaaagaaatgaaaaatgttacatcatggtttcgtgattcaggcttaccacttttcacatttgctcgtgaaagacctttagagttttactttttaatcgctggtggaacatacgaacctcaatacgcaaaatgtagattcttatttacaaaagtagcttgtttacaaactgttttagacgatatgtacgatacttacggtacaccatcagagttaaaattatttactgaggcagttcgtcgttgggatttatcattcacagaaaacttacctgattatatgaaattatgctacaaaatttactatgatattgttcatgaagttgcttgggaagtagaaaaagaacagggacgtgagcttgtttcatttttccgtaaaggttgggaagactatcttttaggttattatgaagaagctgaatggttagctgctgaatacgttcctactttagatgaatacattaaaaacggtattacatctattggtcaacgtattttacttttatcaggtgtacttattatggaaggtcaacttttatcacaagaagctcttgaaaaagtagattatccaggtcgtcgtgttttaacagaattaaacagtttaattagtcgtttagcagacgatactaaaacatacaaagcagaaaaagctcgtggtgaacttgctagtagtattgaatgttatatgaaagaccaccctggttgtcaagaagaagaagcattaaaccatatttatggcattttagaaccagctgttaaagaattaactcgtgagtttcttaaagcagatcacgtaccattcccttgcaaaaaaatgttatttgatgaaacaagagttacaatggtaattttcaaagatggtgatggtttcggtatttctaaattagaagtaaaagaccacataaaagaatgtttaattgagccattaccacttggtaccggtgaaaatctttattttcaaggtagtggtggtggcggttctgactacaaagatgacgacgataaaggaaccggttaatctagactcgag

SEQ ID NO: 20 - DNA, 27 bp, Artificial Sequence
  Primer
gtgctaggtaactaacgtttgattttt

SEQ ID NO: 21 - DNA, 22 bp, Artificial Sequence
  Primer
cgttcttctgagaaatggctta

SEQ ID NO: 22 - DNA, 22 bp, Artificial Sequence
  Primer
gacgttcttgacgttttgtttg

SEQ ID NO: 23 - DNA, 23 bp, Artificial Sequence
  Primer
ggaaggggacgtaggtacataaa

SEQ ID NO: 24 - DNA, 23 bp, Artificial Sequence
  Primer
ttagaacgtgttttgttcccaat

SEQ ID NO: 25 - DNA, 20 bp, Artificial Sequence
  Primer
ccgaactgaggttgggttta

SEQ ID NO: 26 - DNA, 20 bp, Artificial Sequence
  Primer
gggggagcgaataggattag

SEQ ID NO: 27 - DNA, 1005 bp, Artificial Sequence
  Codon optimized sequence
ctcgaggccgccgctgcccccgccgagaccatgaacaagagcgcggcgggcgcggaggtgccggaggcgttcacgagcgtgttccagcccggcaagctggccgtggaggcgatccaggtggacgagaacgcggcccccaccccgcccatccccgtgctgatcgtggccccgaaggacgccggcacctaccccgtggccatgctgctgcacggcttcttcctgcacaaccacttctacgagcacctgctgcgccacgtggccagccacggcttcatcatcgtggccccgcagttcagcatcagcatcatccccagcggcgacgccgaggacatcgccgccgccgccaaggtggccgactggctgcccgacggcctgcccagcgtgctgcccaagggcgtggagccggagctgagcaagctggcgctggccggccactcgcgcggcggccacaccgccttcagcctggccctgggccacgccaagacccagctcaccttcagcgccctgatcggcctggaccccgtggcgggcaccggcaagagcagccagctccagccgaagatcctgacctacgagccgagcagcttcggcatggcgatgcccgtgctggtgatcggcaccggcctgggcgaggagaagaagaacatcttcttcccgccgtgcgccccgaaggacgtgaaccacgcggagttctaccgcgagtgccgcccgccctgctactacttcgtgaccaaggactacggccacctggacatgctggacgacgacgccccgaagttcatcacgtgcgtgtgcaaggacggcaacggctgcaagggcaagatgcgccgctgcgtggcgggcatcatggtggcgttcctgaacgcggccctgggcgagaaggacgccgacctggaggccatcctgcgcgaccccgccgtggcccccaccaccctggacccggtggagcaccgcgtggccaccggcgactacaaggacgacgacgacaagaccggctaaggatcc

(19) United States
(12) Patent Application Publication
(10) Pub. No.: US 2011/0256594 A1
(43) Pub. Date: Oct. 20, 2011

(54) REMOVAL OF NITROGEN FROM A CHLOROPHYLL OR PHEOPHYTIN CONTAINING BIOMASS

(75) Inventors: Richard J. Cranford, San Diego, CA (US); Craig Behnke, San Diego, CA (US); Yan Poon, San Diego, CA (US)

(73) Assignee: SAPPHIRE ENERGY, INC

(21) Appl. No.: 13/133,354

(22) PCT Filed: Dec. 8, 2009
(86) PCT No.: PCT/US09/67222

§ 371 Date: Jun. 30, 2011

Related U.S. Application Data
(60) Provisional application No. 61/120,578, filed on Dec. 8, 2008.

(51) Int. Cl. C12P 7/64; C11B 1/00; C12N 9/00; C10L 1/00

(52) U.S. Cl. 435/134; 44/307; 554/8; 435/183

(57) ABSTRACT

The present disclosure relates to refining a product from a biomass containing chlorophyll and/or pheophytins. In particular, a method of refining a product (such as a biofuel) from a photosynthetic organism is disclosed. The photosynthetic organism can be a naturally occurring organism or a genetically modified or altered organism. The method of refining comprises removing nitrogen to obtain the desired product. In some aspects, nitrogen is removed from a chlorophyll and/or pheophytin containing product by enzymatic degradation of chlorophyll and/or pheophytins and subsequent removal of the nitrogen

### CROSS REFERENCE TO RELATED APPLICATION

This application claims the benefit of U.S. Provisional Application No. 60/120,578, filed Dec. 8, 2008, the entire contents of which are incorporated by reference for all purposes.

### INCORPORATION BY REFERENCE

All publications, patents, patent applications, public databases, public database entries, and other references cited in this application are herein incorporated by reference in their entirety as if each individual publication, patent, patent application, public database, public database entry, or other reference was specifically and individually indicated to be incorporated by reference.

### BACKGROUND

Photosynthetic microorganisms, both naturally occurring and genetically modified or altered, may provide a means to produce products, for example biofuels or pharmaceutical products. However, such products often contain chlorophyll and/or pheophytin, rich sources of nitrogen. Thus, when photosynthetic organisms are used to produce such products, removal of nitrogen contaminates from the product preparations may be desired. Thus, a need exists for improved methods to conveniently and economically produce more refined bio-products. Disclosed herein are novel compositions and methods used for producing a refined product (e.g. a bio-product) from both naturally occurring and genetically modified or altered photosynthetic organisms.

### SUMMARY

Provided herein are methods for producing a nitrogen-depleted product from a photosynthetic organism comprising, obtaining a biomass composition from the photosynthetic organism wherein the biomass composition comprises one or more chlorophylls and/or one of more pheophytins and a product of interest, degrading at least a subset of the chlorophyll or pheophytin in the biomass composition, removing a cleaved portion of the degraded chlorophyll or pheophytin wherein the cleaved portion comprises nitrogen, and refining said biomass composition after the removal step to produce the nitrogen-depleted product.

The biomass composition may be a wet biomass composition or a dry or semi-dry biomass composition. The biomass composition may comprise a lysate of the photosynthetic organism.

The degrading step may comprise hydrolysis, alcoholysis, glycolysis, or cleavage in an anhydrous environment or an aqueous environment. The degrading step may comprise the use of one or more enzymes. In one embodiment, the enzyme, for example, is a chlorophyllase. The degrading step may comprise the use of one or more acids. In other embodiments, the acid may be an organic acid or an inorganic acid. In other embodiments, the acid may be hydrochloric acid, citric acid, nitric acid, acetic acid, sulfuric acid, formic acid, phosphoric acid, succinic acid, or a solid acid catalyst. In other embodiments, the solid acid catalyst may be an acidified aluminum oxide, acidified silicon dioxide, acidified zironium hydroxide, acidified zeolite, or activated carbon. The degrading step may comprise the use of one or more bases. In other embodiments, the base may be bleach, sodium hydroxide, potassium hydroxide, ammonia, sodium carbonate, calcium carbonate, calcium hydroxide, or a solid base catalyst. In other embodiments, the solid base catalyst may be calcium methoxide, calcium oxide, potassium hydroxide/aluminum oxide, or magnesium oxide. The degrading step may comprise heating the biomass composition. In other embodiments, the biomass may be heated to 25° C. to 95° C., 30° C. to 60° C., 37° C. to 95° C., 60° C. to 250° C., or 80° C. to 200° C. In other embodiments, the biomass is heated to 120° C. In other embodiments, the biomass may be heated to up to 25° C., 30° C., 35° C., 40° C., 45° C., 50° C., 55° C., 60° C., or 65° C. The degrading step may comprise cooling the biomass composition. In other embodiments, the biomass may be cooled to less than 0 to −40° C. or −5° C. to −20° C. In another embodiment, the biomass is cooled to less than 25° C. In yet another embodiment, the biomass is cooled to less than −20° C. The degrading step may further comprise the addition of one or more glycerol, acetone, non-ionic surfactant, detergent, or divalent cations, to the biomass composition. The degrading step may be at pH 6.5 to pH 12. Prior to the degrading step, the biomass composition may comprise at least 0.5%, 1%, 2%, 3%, 4%, 5%, 6%, 7%, or 8% chlorophyll (w/w).

The cleaved portion that is removed may be chlorophyllide. In other embodiments, the chlorophyllide may be removed by dissolving or dispersing the biomass composition in one or more solvents. In other embodiments, the solvent may be water, acetone, glycerol, alcohol, hexane, heptane, methylpentane, toluene, or methylisobutylketone. In other embodiments, the alcohol may be methanol, propanol, ethanol, or isopropanol. In another embodiment, the solvent is an oil immiscible solvent.

The cleaved portion that is removed may be pheophorbide. In other embodiments, the pheophorbide may be removed by dissolving or dispersing the biomass composition in one or more solvents. In other embodiments, the solvent may be water, acetone, glycerol, alcohol, hexane, heptane, methylpentane, toluene, or methylisobutylketone. In other embodiments, the alcohol may be methanol, propanol, ethanol, or isopropanol. In another embodiment, the solvent is an oil immiscible solvent.

The removing step may comprise adding one or more solvents and may comprise removing the one or more solvents. In other embodiments, the solvent may be water, acetone, glycerol, alcohol, hexane, heptane, methylpentane, toluene, or methylisobutylketone. In other embodiments, the alcohol may be methanol, propanol, ethanol, or isopropanol. In another embodiment, the solvent is an oil immiscible solvent. The removing step may comprise a filtering step. The removing step may not comprise the addition of an adsorbent material. The removing step may not comprise the addition of an adsorbent material, wherein the adsorbent material is bleaching clay or a carbonaceous material. The removing step may not comprise adsorption of the nitrogen on to a solid support solid, wherein the solid support is a nanomaterial or bleaching clay. The removing step may comprise dissolving a nitrogen containing pigment in an oil immiscible solvent. The removing step may remove substantially all nitrogen to result in a nitrogen-depleted product substantially free of nitrogen. In other embodiments, the nitrogen-depleted product may contain up to 0.1%, 0.08%, 0.06%, 0.04%, 0.02%, 0.01%, 0.008%, 0.006%, 0.004%, or 0.002% nitrogen (w/w). After the removing step, the biomass composition may comprise up to 1% (w/w) chlorophyll.

The refining step may comprise drying, crushing/lysis, extraction, evaporation, cracking, heating, cooling, mixing, holding, hydrating, washing, extracting, filtering, drying, distillation, bleaching, deodorization, degumming, decanting, fractionation, separating, phase separation, sediment removal by any means or centrifugation, or a combination of any two or more of the above processes. The refining step may comprise the removal of one or more phosphorus, trace metals, trace heteroatoms, or residual nitrogens. The methods described above, may further comprise hydrogenation of the nitrogen-depleted product. In another embodiment, the methods described above, may further comprise cracking of the nitrogen-depleted product.

The biomass composition may comprise pigments from the photosynthetic organism. The photosynthetic organism may be genetically modified to produce a fatty acid, lipid, or hydrocarbon. In another embodiment, the photosynthetic organism is genetically modified to produce a chlorophyllase. The photosynthetic organism may be a prokaryote. In one embodiment, the prokaryote may be a cyanobacterium. In another embodiment, the photosynthetic organism may be a eukaryote. In yet another embodiment, the eukaryote may be a vascular plant. In another embodiment, the eukaryote may be a non-vascular photosynthetic organism. In one embodiment, the non-vascular photosynthetic organism may be an alga. In yet another embodiment, the alga may be a green alga. In another embodiment, the green alga may be a Chlorophycean. In other embodiments, the green alga may be a Chlamydomonas, Scenedesmus, Chlorella or Nannochlorpis. In one embodiment, the Chlamydomonas is C. reinhardtii. In another embodiment, the Chlamydomonas is C. reinhardtii 137c.

The methods described above may comprise an additional step wherein the degrading of at least a subset of the chlorophyll or pheophytin in the biomass composition is conducted twice.

The nitrogen-depleted product may comprise one or more fatty acids, lipids, or hydrocarbons. In other embodiments, the acid, lipid, or hydrocarbon is not naturally found in the photosynthetic organism. The nitrogen-depleted product may comprise one or more hydrocarbons. In one embodiment, the hydrocarbon may be an isoprenoid. In other embodiments, the isoprenoid may be a monoterpene, sesquiterpene, diterpene, sesterpene, triterpene, carotenoid, squalene, or a neophytadiene. The nitrogen-depleted product may comprise one or more phytol, phytadiene, or neophytadiene. The nitrogen-depleted product may be a biofuel. The nitrogen-depleted product may be an oil. The nitrogen-depleted product may comprise one or more neutral lipids. In other embodiments, the neutral lipid may be a fatty acid, carotenoid, fatty alcohol, sterol, triglyceride, wax ester, or sterol ester. The nitrogen-depleted product may comprises about 5% to about 95% free fatty acids (w/w), about 10% to about 90% free fatty acids (w/w), about 50% to about 85% free fatty acids (w/w), or about 85% free fatty acids (w/w). Heteroatoms may be removed from the nitrogen-depleted product. In other embodiments, the heteroatoms may be one or more of oxygen, phosphorus, nitrogen, sulfur, or metals. The nitrogen may be contained in a pigment.

The methods described above may further comprise, hydrolyzing chlorophyll or pheophytin after degrading at least a subset of the chlorophyll or pheophytin in the biomass composition.

Another aspect provides nitrogen-depleted products produced by any of the methods described above. Yet another aspect provides, nitrogen-depleted products comprising about 5% to about 95% free fatty acids (w/w), about 10% to about 90% free fatty acids (w/w), about 50% to about 85% free fatty acids (w/w), or about 85% free fatty acids (w/w). Another aspect provides, nitrogen-depleted products wherein heteroatoms may be removed from the nitrogen-depleted product. In other embodiments, the heteroatoms are one or more of oxygen, phosphorus, nitrogen, sulfur, or metals.

Also provided herein are methods for producing a nitrogen-depleted product from a photosynthetic organism comprising: removing, without the use of a filter, nitrogen from a composition comprising the photosynthetic organism; and refining the remainder of the composition to produce the nitrogen-depleted product.

The composition may be a wet biomass composition or a dry or semi-dry composition. The composition may comprise a lysate of the photosynthetic organism.

The removing step may comprise adding one or more solvents and may comprise removing the one or more solvents. In other embodiments, the solvent may be water, acetone, glycerol, alcohol, hexane, heptane, methylpentane, toluene, or methylisobutylketone. In other embodiments, the alcohol may be methanol, propanol, ethanol, or isopropanol. In another embodiment, the solvent is an oil immiscible solvent.

The removing step may not comprise the addition of an adsorbent material. The removing step may not comprise the addition of an adsorbent material, wherein the adsorbent material is bleaching clay or a carbonaceous material. The removing step may not comprise adsorption of the nitrogen on to a solid support solid, wherein the solid support is a nanomaterial or bleaching clay.

The removing step may comprise dissolving a nitrogen containing pigment in an oil immiscible solvent. The removing step may remove substantially all nitrogen to result in a nitrogen-depleted product substantially free of nitrogen. In other embodiments, the nitrogen-depleted product may contain up to 0.1%, 0.08%, 0.06%, 0.04%, 0.02%, 0.01%, 0.008%, 0.006%, 0.004%, or 0.002% nitrogen (w/w). After the removing step, the biomass composition may comprise up to 1% (w/w) chlorophyll.

The refining step may comprise drying, crushing/lysis, extraction, evaporation, cracking, heating, cooling, mixing, holding, hydrating, washing, extracting, filtering, drying, distillation, bleaching, deodorization, degumming, decanting, fractionation, separating, phase separation, sediment removal by any means or centrifugation, or a combination of any two or more of the above processes. The refining step may comprise the removal of one or more phosphorus, trace metals, trace heteroatoms, or residual nitrogens.

The methods described above, may further comprise hydrogenation of the nitrogen-depleted product. In another embodiment, the methods described above, may further comprise cracking of the nitrogen-depleted product.

The biomass composition may comprise pigments from the photosynthetic organism. The photosynthetic organism may be genetically modified to produce a fatty acid, lipid, or hydrocarbon. In another embodiment, the photosynthetic organism is genetically modified to produce a chlorophyllase. The photosynthetic organism may be a prokaryote. In one embodiment, the prokaryote may be a cyanobacterium. In another embodiment, the photosynthetic organism may be a eukaryote. In yet another embodiment, the eukaryote may be a vascular plant. In another embodiment, the eukaryote may be a non-vascular photosynthetic organism. In one embodiment, the non-vascular photosynthetic organism may be an alga. In yet another embodiment, the alga may be a green alga. In another embodiment, the green alga may be a Chlorophycean. In other embodiments, the green alga may be a Chlamydomonas, Scenedesmus, Chlorella or Nannochlorpis. In one embodiment, the Chlamydomonas is C. reinhardtii. In another embodiment, the Chlamydomonas is C. reinhardtii 137c.

The nitrogen-depleted product may comprise one or more fatty acids, lipids, or hydrocarbons. In other embodiments, the acid, lipid, or hydrocarbon is not naturally found in the photosynthetic organism. The nitrogen-depleted product may comprise one or more hydrocarbons. In one embodiment, the hydrocarbon may be an isoprenoid. In other embodiments, the isoprenoid may be a monoterpene, sesquiterpene, diterpene, sesterpene, triterpene, carotenoid, squalene, or a neophytadiene. The nitrogen-depleted product may comprise one or more phytol, phytadiene, or neophytadiene. The nitrogen-depleted product may be a biofuel. The nitrogen-depleted product may be an oil. The nitrogen-depleted product may comprise one or more neutral lipids. In other embodiments, the neutral lipid may be a fatty acid, carotenoid, fatty alcohol, sterol, triglyceride, wax ester, or sterol ester. The nitrogen-depleted product may comprises about 5% to about 95% free fatty acids (w/w), about 10% to about 90% free fatty acids (w/w), about 50% to about 85% free fatty acids (w/w), or about 85% free fatty acids (w/w). Heteroatoms may be removed from the nitrogen-depleted product. In other embodiments, the heteroatoms may be one or more of oxygen, phosphorus, nitrogen, sulfur, or metals. The nitrogen may be contained in a pigment.

Another aspect provides nitrogen-depleted products produced by any of the methods described above. Yet another aspect provides, nitrogen-depleted products comprising about 5% to about 95% free fatty acids (w/w), about 10% to about 90% free fatty acids (w/w), about 50% to about 85% free fatty acids (w/w), or about 85% free fatty acids (w/w). Another aspect provides, nitrogen-depleted products wherein heteroatoms may be removed from the nitrogen-depleted product. In other embodiments, the heteroatoms are one or more of oxygen, phosphorus, nitrogen, sulfur, or metals.

Provided herein are methods for producing a nitrogen-depleted oil from a non-vascular photosynthetic organism comprising: obtaining a biomass composition from the non-vascular photosynthetic organism wherein the biomass composition comprises one or more chlorophylls and/or one or more pheophytins and an oil of interest; adding an enzyme to the biomass composition; removing nitrogen from the biomass composition; and, refining the remainder of the composition to produce the nitrogen-depleted oil.

The biomass composition may be a wet biomass composition or a dry or semi-dry biomass composition. The biomass composition may comprise a lysate of the photosynthetic organism.

In one embodiment, the enzyme, for example, is a chlorophyllase.

The removing step may comprise adding one or more solvents and may comprise removing the one or more solvents. In other embodiments, the solvent may be water, acetone, glycerol, alcohol, hexane, heptane, methylpentane, toluene, or methylisobutylketone. In other embodiments, the alcohol may be methanol, propanol, ethanol, or isopropanol. In another embodiment, the solvent is an oil immiscible solvent. The removing step may comprise a filtering step. The removing step may not comprise the addition of an adsorbent material. The removing step may not comprise the addition of an adsorbent material, wherein the adsorbent material is bleaching clay or a carbonaceous material. The removing step may not comprise adsorption of the nitrogen on to a solid support solid, wherein the solid support is a nanomaterial or bleaching clay. The removing step may comprise dissolving a nitrogen containing pigment in an oil immiscible solvent. The removing step may remove substantially all nitrogen to result in a nitrogen-depleted product substantially free of nitrogen. In other embodiments, the nitrogen-depleted product may contain up to 0.1%, 0.08%, 0.06%, 0.04%, 0.02%, 0.01%, 0.008%, 0.006%, 0.004%, or 0.002% nitrogen (w/w). After the removing step, the biomass composition may comprise up to 1% (w/w) chlorophyll.

The refining step may comprise drying, crushing/lysis, extraction, evaporation, cracking, heating, cooling, mixing, holding, hydrating, washing, extracting, filtering, drying, distillation, bleaching, deodorization, degumming, decanting, fractionation, separating, phase separation, sediment removal by any means or centrifugation, or a combination of any two or more of the above processes. The refining step may comprise the removal of one or more phosphorus, trace metals, trace heteroatoms, or residual nitrogens.

The methods described above, may further comprise hydrogenation of the nitrogen-depleted product. In another embodiment, the methods described above, may further comprise cracking of the nitrogen-depleted product.

The biomass composition may comprise pigments from the photosynthetic organism. The photosynthetic organism may be genetically modified to produce a fatty acid, lipid, or hydrocarbon. In another embodiment, the photosynthetic organism is genetically modified to produce a chlorophyllase.

In one embodiment, the non-vascular photosynthetic organism may be an alga. In yet another embodiment, the alga may be a green alga. In another embodiment, the green alga may be a Chlorophycean. In other embodiments, the green alga may be a Chlamydomonas, Scenedesmus, Chlorella or Nannochlorpis. In one embodiment, the Chlamydomonas is C. reinhardtii. In another embodiment, the Chlamydomonas is C. reinhardtii 137c.

The nitrogen-depleted oil may comprise one or more fatty acids, lipids, or hydrocarbons. In other embodiments, the acid, lipid, or hydrocarbon is not naturally found in the photosynthetic organism. The nitrogen-depleted oil may comprise one or more hydrocarbons. In one embodiment, the hydrocarbon may be an isoprenoid. In other embodiments, the isoprenoid may be a monoterpene, sesquiterpene, diterpene, sesterpene, triterpene, carotenoid, squalene, or a neophytadiene. The nitrogen-depleted oil may comprise one or more phytol, phytadiene, or neophytadiene. The nitrogen-depleted oil may comprise one or more neutral lipids. In other embodiments, the neutral lipid may be a fatty acid, carotenoid, fatty alcohol, sterol, triglyceride, wax ester, or sterol ester. The nitrogen-depleted oil may comprises about 5% to about 95% free fatty acids (w/w), about 10% to about 90% free fatty acids (w/w), about 50% to about 85% free fatty acids (w/w), or about 85% free fatty acids (w/w). Heteroatoms may be removed from the nitrogen-depleted oil. In other embodiments, the heteroatoms may be one or more of oxygen, phosphorus, nitrogen, sulfur, or metals. The nitrogen may be contained in a pigment.

Another aspect provides nitrogen-depleted oil produced by any of the methods described above. Yet another aspect provides, nitrogen-depleted oil comprising about 5% to about 95% free fatty acids (w/w), about 10% to about 90% free fatty acids (w/w), about 50% to about 85% free fatty acids (w/w), or about 85% free fatty acids (w/w). Another aspect provides, nitrogen-depleted oil wherein heteroatoms may be removed from the nitrogen-depleted product. In other embodiments, the heteroatoms are one or more of oxygen, phosphorus, nitrogen, sulfur, or metals.

Also provided herein are methods for producing a nitrogen-depleted bio-fuel from a non-vascular photosynthetic organism comprising: obtaining a biomass composition from the non-vascular photosynthetic organism wherein the biomass composition comprises one or more chlorophylls and/or one or more pheophytins and an oil of interest; adding a chlorophyllase to the biomass composition; removing nitrogen containing pigments from the biomass composition; and, cracking the nitrogen-depleted composition to produce the bio-fuel.

The biomass composition may be a wet biomass composition or a dry or semi-dry biomass composition. The biomass composition may comprise a lysate of the photosynthetic organism.

The removing step may comprise adding one or more solvents and may comprise removing the one or more solvents. In other embodiments, the solvent may be water, acetone, glycerol, alcohol, hexane, heptane, methylpentane, toluene, or methylisobutylketone. In other embodiments, the alcohol may be methanol, propanol, ethanol, or isopropanol. In another embodiment, the solvent is an oil immiscible solvent. The removing step may comprise a filtering step. The removing step may not comprise the addition of an adsorbent material. The removing step may not comprise the addition of an adsorbent material, wherein the adsorbent material is bleaching clay or a carbonaceous material. The removing step may not comprise adsorption of the nitrogen on to a solid support solid, wherein the solid support is a nanomaterial or bleaching clay. The removing step may comprise dissolving a nitrogen containing pigment in an oil immiscible solvent. The removing step may remove substantially all nitrogen to result in a nitrogen-depleted product substantially free of nitrogen. In other embodiments, the nitrogen-depleted product may contain up to 0.1%, 0.08%, 0.06%, 0.04%, 0.02%, 0.01%, 0.008%, 0.006%, 0.004%, or 0.002% nitrogen (w/w). After the removing step, the biomass composition may comprise up to 1% (w/w) chlorophyll.

The photosynthetic organism may be genetically modified to produce a fatty acid, lipid, or hydrocarbon. In another embodiment, the photosynthetic organism is genetically modified to produce a chlorophyllase.

In one embodiment, the non-vascular photosynthetic organism may be an alga. In yet another embodiment, the alga may be a green alga. In another embodiment, the green alga may be a Chlorophycean. In other embodiments, the green alga may be a Chlamydomonas, Scenedesmus, Chlorella or Nannochlorpis. In one embodiment, the Chlamydomonas is C. reinhardtii. In another embodiment, the Chlamydomonas is C. reinhardtii 137c.

The bio-fuel may comprise one or more fatty acids, lipids, or hydrocarbons. In other embodiments, the acid, lipid, or hydrocarbon is not naturally found in the photosynthetic organism. The bio-fuel may comprise one or more hydrocarbons. In one embodiment, the hydrocarbon may be an isoprenoid. In other embodiments, the isoprenoid may be a monoterpene, sesquiterpene, diterpene, sesterpene, triterpene, carotenoid, squalene, or a neophytadiene. The bio-fuel may comprise one or more phytol, phytadiene, or neophytadiene. The bio-fuel may comprise one or more neutral lipids. In other embodiments, the neutral lipid may be a fatty acid, carotenoid, fatty alcohol, sterol, triglyceride, wax ester, or sterol ester.

Another aspect provides bio-fuels produced by any of the methods described above.

Another aspect provides compositions comprising a bio-fuel, phytol, and up to about 0.5% (w/w) chlorophyll, chlorophyllide, pheophorbide, or pheophytin. In one embodiment, the phytol is at least about 1% (w/w) of the composition. In another embodiment, the composition comprises one or more isoprenoids. In other embodiments, the one or more isoprenoids is a monoterpene, sesquiterpene, diterpene, sesterpene, triterpene, carotenoid, squalene or neophytadiene. In another embodiment, the composition further comprises pigments from a photosynthetic organism. In yet another embodiment, the pigments are derived from algae.

Also provided is a composition comprising up to about 75% (w/w) free fatty acids and up to about 10% (w/w) phytol. Also provided is a composition comprising up to about 50% (w/w) free fatty acids and up to about 10% (w/w) phytol. Also provided is a composition comprising up to about 85% (w/w) free fatty acids and up to about 10% (w/w) phytol. Also provided is a composition comprising up to about 25% (w/w) free fatty acids, up to about 25% triglycerids, and up to about 15% (w/w) wax esters. Also provided are compositions comprising up to about 40% (w/w) free fatty acids, up to about 40% triglycerids, and up to about 40% (w/w) wax esters, wherein the total % (w/w) cannot exceed 100%.

Disclosed herein is a method for producing a nitrogen-depleted product from a photosynthetic organism comprising (a) obtaining a biomass composition from the photosynthetic organism wherein the biomass composition comprises one or more chlorophylls and a product of interest, (b) degrading at least a subset of the chlorophyll in the biomass composition, (c) removing a cleaved portion of the degraded chlorophyll wherein the cleaved portion comprises nitrogen; and (d) refining said biomass composition after the removal step to produce the nitrogen-depleted product of interest. In some instances, the degrading step may occur in an anhydrous environment. In one example the degrading step comprises hydrolysis, alcoholysis or glycolysis. In another example the refining step comprises drying, crushing/lysis, evaporation, cracking, heating, cooling, mixing, holding, hydrating, washing, extracting, filtering, drying, distillation, bleaching, deodorization, degumming, decanting, fractionation, separating, phase separation, and sediment removal by any means or centrifugation. In some aspects the remaining composition comprises phytol. In another aspect the organism is a non-vascular photosynthetic organism. In one example the degrading step utilizes an enzyme. The enzyme can be a chlorophyllase. In some aspects the cleaved portion removed is chlorophyllide. In some aspects the removing step comprises adding a solvent and removing a solvent. The solvent can be water, acetone, glycerol, alcohol, hexane, heptane, methylpentane, toluene, or methylisobutylketone. If the solvent is an alcohol, examples of alchohols include, but are not limited to, methanol, propanol, ethanol, and isopropanol. In some aspects the composition comprises pigments from a photosynthetic organism. In some aspects the removing step does not use a filtering step. In some aspects a second cleaved portion of the chlorophyll is also removed from the composition. The second cleaved portion can be phytol. In some aspects the removing step removes substantially all nitrogen to result in a product substantially free of nitrogen. The nitrogen depleted product can comprise one or more isoprenoids. The isoprenoids can be a monoterpene, sesquiterpene, diterpene, sesterpene, triterpene, carotenoid, squalene or neophytadiene. In some aspects prior to the degrading step, the biomass comprises at least about 5% chlorophyll (w/w). In some aspects after the removal step the biomass comprises up to about 1% (w/w) chlorophyll. In some aspects the biomass comprises a lysate of the organism.

One aspect relates to a method of producing a nitrogen-depleted product from a photosynthetic organism comprising: removing, without the use of a filter, nitrogen from a composition comprising the photosynthetic organism; and refining the remainder of the composition to produce the nitrogen-depleted product. In some aspects the nitrogen-depleted product comprises a fatty acid, lipid or hydrocarbon. In some aspects the filter is an adsorbent material. In some aspects the adsorbent material is bleaching clay or a carbonaceous material. In some aspects the removing step comprises hydrolyzing chlorophyll. The method may further comprise dissolving chlorophyllide in a solvent. The solvent can be water, acetone, glycerol, alcohol, hexane, heptane, methylpentane, toluene, or methylisobutylketone. If the solvent is an alcohol, examples of alchohols include, but are not limited to, methanol, propanol, ethanol, and isopropanol. In some aspects the removing step comprises adding an enzyme. The enzyme can be chlorophyllase. In some aspects the nitrogen-depleted product comprises one or more hydrocarbons. The hydrocarbon can be an isoprenoid. The isoprenoid can be a monoterpene, sesquiterpene, diterpene, sesterpene, triterpene, carotenoid, squalene or neophytadiene. In some aspects the composition depleted of nitrogen comprises phytol. In some aspect the refining comprises cracking or hydrogenation of the product. In some aspects the organism is algae or cyanobacteria.

In an aspect, a method is disclosed for producing a nitrogen-depleted oil from a non-vascular photosynthetic organism comprising: obtaining a biomass composition from the non-vascular photosynthetic organism wherein the biomass composition comprises one or more chlorophylls and an oil of interest; adding an enzyme to the biomass composition; removing nitrogen from the biomass composition; and refining the remainder of the composition to produce the nitrogen-depleted oil. In some instances, the oil of interest comprises a fatty acid, lipid or hydrocarbon. An oil of interest can comprise a fatty acid, lipid or hydrocarbon not naturally found in the non-vascular photosynthetic organism. In some instances, the non-vascular photosynthetic organism is genetically altered to produce a fatty acid, lipid or hydrocarbon. In an embodiment, the enzyme is a chlorophyllase. In some instances, the removing process does not comprise adsorption of the nitrogen on to a solid support solid, for example, a nanomaterial or bleaching clay. In other instances, the removing process does not comprise precipitation of the nitrogen and the precipitation of nitrogen comprises precipitation of chlorophyll. In some instances, the removing process comprises dissolving nitrogen containing pigments into an oil immiscible solvent. The removing process can comprise removal of chlorophyllide or pheophorbide. The removing process can comprise dissolving a chlorophyllide or a pheophorbide in an oil immiscible solvent. In other embodiments, the solvent can be water, acetone, glycerol, alcohol, hexane, heptane, methylpentane, toluene, or methylisobutylketone. If the solvent is an alcohol, examples of alchohols include, but are not limited to, methanol, propanol, ethanol, and isopropanol. In some instances, the nitrogen is contained in a pigment. The nitrogen-depleted oil can comprise one or more hydrocarbons and the hydrocarbon can be an isoprenoid. The isoprenoid can be a monoterpene, sesquiterpene, diterpene, sesterpene, triterpene, carotenoid, squalene or neophytadiene. In some instances, the nitrogen-depleted oil comprises phytol. A method as described can further comprise hydrolyzing chlorophyll after the step of adding and an enzyme. A method can also further comprise a cracking step to refine the oil into a biofuel.

In another aspect disclosed herein, a method of producing a nitrogen-depleted bio-fuel from a non-vascular photosynthetic organism comprises: obtaining a biomass composition from the non-vascular photosynthetic organism wherein the biomass composition comprises one or more chlorophylls and an oil of interest; adding a chlorophyllase to the biomass composition; removing nitrogen containing pigments from the biomass composition; and cracking the nitrogen-depleted composition to produce the bio-fuel is disclosed.

Disclosed herein is a composition comprising a bio-fuel, phytol and up to about 0.5% (w/w) chlorophyll or chlorophyllide. In some aspects the volume of the composition is greater than 500 liters. In some aspects the phytol is at least about 1% (w/w) of said composition. In some aspects said composition comprises one or more isoprenoids. The one or more isoprenoids can be a monoterpene, sesquiterpene, diterpene, sesterpene, triterpene, carotenoid, squalene or neophytadiene. In some aspects the composition further comprises pigments from a photosynthetic organism. In some aspects the pigments are derived from algae.

### BRIEF DESCRIPTION OF THE DRAWINGS

These and other features, aspects, and advantages of the present disclosure will become better understood with regard to the following description, appended claims and accompanying figures where:

FIG. 1 illustrates two constructs for insertion of a gene into a chloroplast genome.

FIG. 2 illustrates primer pairs for PCR screening of transformants and expected band profiles for wild-type, heteroplasmic and homoplasmic strains.

FIG. 3 illustrates results from PCR screening and Western blot analysis of endo-β-glucanase transformed C. reinhardtii clones.

FIG. 4 is a graphic representation of additional nucleic acid constructs.

FIG. 5 shows PCR and Western analysis of C. reinhardtii transformed with FPP synthase and bisabolene synthase.

FIG. 6 shows gas chromatography-mass spectrometry analysis of C. reinhardtii transformed with FPP synthase and bisabolene synthase.

FIG. 7 is a flow diagram outlining one non-limiting example of a refining method and one non-limiting example of integrating a nitrogen removal process into a refining method.

FIG. 8 is a flow diagram illustrating additional examples of integrating a nitrogen removal process into a refining method.

FIG. 9 is a flow diagram illustrating additional examples of integrating a nitrogen removal process into a refining method.

FIG. 10 is a flow diagram illustrating additional examples of integrating a nitrogen removal process into a refining method.

FIG. 11 is a photograph of a thin layer chromatography (TLC) plate illustrating how pheophytin was removed from product oil from the KOH pretreated biomass but not from biomass without the KOH pretreatment.

FIG. 12 shows gas chromatograms for two oils from identical biomass samples: one oil sample is from base hydrolysed biomass (lighter line) and the other oil sample is from biomass that was not base hydrolysed (darker line).

FIG. 13 illustrates a construct for insertion of a gene into a chloroplast genome.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

As used in this specification and the appended claims, the singular forms “a”, “an” and “the” include plural reference unless the context clearly dictates otherwise.

The present disclosure relates to methods for producing a product (e.g., fuel product) using a photosynthetic organism, such as a vascular or non-vascular photosynthetic organism (NVPO). In some instances the product is endogenously or naturally produced by an algae or cyanobacteria, e.g., Chlamydomonas, Scenedesmus, Chlorella or Nannochlorpis. Such organisms can be genetically modified or altered to produce a product not naturally produced by the organism, or to increase production of a product that it is naturally produced by the organism, or both.

In some instances an organism herein is modified to increase production of non-native occurring lipids including, but not limited to fatty acids, lipids or hydrocarbons such as acylglycerols, aldehydes, amino compound-containing lipids, amino alcohols, ceramides, cyanolipids, fatty alcohols, ketones, phenolic lipids, prostinoids and related compounds, quinones, steroids, sterols, terpenoids, vitamin alcohols, carotenoids and waxes.

The photosynthetic organism can be transformed with one or more nucleic acids to facilitate the production of lipids such as fatty acids, hydrocarbons, etc. (e.g. a nucleic acid that directs the expression of an enzyme). Exogenous nucleic acids can be introduced into the photosynthetic organisms (e.g. into the chloroplast) by any suitable method to generate a modified photosynthetic organism. The photosynthetic organism can be transfected or transformed (i.e. genetically modified) with at least one nucleic acid encoding one or more proteins (e.g. enzymes). A single genetically modified photosynthetic organism can comprise exogenous nucleic acids encoding one, two, three or more proteins or subunits thereof (e.g. C. reinhardtii can be genetically modified to produce both an endoxylanase and an endo-β-glucanase). The photosynthetic organism can be genetically modified to contain multiple copies of a nucleic acid that encodes the same protein. The photosynthetic organism can be engineered to contain one or more nucleic acids with one or more mutations. The nucleic acids can comprise a plastid promoter or a nuclear promoter to direct expression in the nucleus, in the chloroplast or plastid of the host organism. The nucleic acid (e.g. vector) may also encode a fusion protein or agent that selectively targets the expressed protein of interest to the nucleus, the chloroplast or plastid.

Nucleic acids can be contained in vectors, including cloning and expression vectors. A cloning vector is a self-replicating DNA molecule that serves to transfer a DNA segment into a host cell. The three most common types of cloning vectors are bacterial plasmids, phages, and other viruses. An expression vector is a cloning vector designed so that a coding sequence inserted at a particular site will be transcribed and translated into a protein.

Both cloning and expression vectors contain nucleotide sequences that allow the vectors to replicate in one or more suitable host cells. In cloning vectors, this sequence is generally one that enables the vector to replicate independently of the host cell chromosomes, and also includes either origins of replication or autonomously replicating sequences. Various bacterial and viral origins of replication are well known to those skilled in the art and include, but are not limited to the pBR322 plasmid origin, the 2u plasmid origin, and the SV40, polyoma, adenovirus, VSV and BPV viral origins.

In some instances, the vectors will contain elements such as an E. coli or S. cerevisiae origin of replication. Such features, combined with appropriate selectable markers, allows for the vector to be “shuttled” between the target host cell and the bacterial and/or yeast cell. The ability to passage a shuttle vector in a secondary host may allow for more convenient manipulation of the features of the vector. For example, a reaction mixture containing the vector and putative inserted polynucleotides encoding the protein can be transformed into prokaryote host cells such as E. coli, amplified and collected using routine methods, and examined to identify vectors containing an insert or construct of interest. If desired, the vector can be further manipulated, for example, by performing site directed mutagenesis of the inserted polynucleotide, then again amplifying and selecting vectors having a mutated polynucleotide of interest. A shuttle vector then can be introduced into plant cell chloroplasts, wherein a polypeptide of interest can be expressed and, if desired, isolated according to a method of the invention.

The nucleic acid sequences may be used inserted into expression vectors. Suitable expression vectors include chromosomal, non-chromosomal and synthetic DNA sequences, for example, SV 40 derivatives; bacterial plasmids; phage DNA; baculovirus; yeast plasmids; vectors derived from combinations of plasmids and phage DNA; and viral DNA such as vaccinia, adenovirus, fowl pox virus, and pseudorabies. In addition, any other vector that is replicable and viable in the host may be used.

The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

A vector can also contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, sequences that encode a selectable marker, and the like. As such, the vector can contain, for example, one or more cloning sites such as a multiple cloning site, which can, but need not, be positioned such that a heterologous polynucleotide can be inserted into the vector and operatively linked to a desired element. The vector also can contain a prokaryote origin of replication (ori), for example, an E. coli ori or a cosmid ori, thus allowing passage of the vector in a prokaryote host cell, as well as in a plant chloroplast, as desired.

A regulatory element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, and an IRES. Typically, a regulatory element includes a promoter and transcriptional and translational stop signals. Elements may be provided with linkers for the purpose of introducing specific restriction sites facilitating ligation of the control sequences with the coding region of a nucleotide sequence encoding a polypeptide. In some instances, such vectors include promoters. Additionally, a cell compartmentalization signal (i.e., a sequence that targets a polypeptide to the cytosol, nucleus, chloroplast membrane or cell membrane). Such signals are well known in the art and have been widely reported (see, e.g., U.S. Pat. No. 5,776,689). A regulatory region, as the term is used herein, can comprise any one or more regulatory elements described above.

In an expression vector, the sequence of interest is operably linked to a suitable expression control sequence or promoter recognized by the host cell to direct mRNA synthesis. Promoters are untranslated sequences located generally 100 to 1000 base pairs (bp) upstream from the start codon of a structural gene that regulate the transcription and translation of nucleic acid sequences under their control. Promoters are generally classified as either inducible or constitutive. Inducible promoters are promoters that initiate increased levels of transcription from DNA under their control in response to some change in the environment, e.g. the presence or absence of a nutrient or a change in temperature. Constitutive promoters, in contrast, maintain a relatively constant level of transcription. Examples of inducible promoters/regulatory elements include, for example, a nitrate-inducible promoter (Bock et al, Plant Mol. Biol. 17:9 (1991)), or a light-inducible promoter, (Feinbaum et al, Mol. Gen. Genet. 226:449 (1991); Lam and Chua, Science 248:471 (1990)), or a heat responsive promoter (Muller et al., Gene 111: 165-73 (1992)).

A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading phase. Linking is achieved by ligation at restriction enzyme sites. If suitable restriction sites are not available, then synthetic oligonucleotide adapters or linkers can be used as is known to those skilled in the art. Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

Common promoters used in expression vectors include, but are not limited to, LTR or SV40 promoters, the E. coli lac or trp promoters, and the phage lambda PL promoter. Other promoters known to control the expression of genes in prokaryotic or eukaryotic cells can be used and are known to those skilled in the art. Non-limiting examples of promoters are endogenous promoters such as the psbA and atpA promoter. Expression vectors may also contain a ribosome binding site for translation initiation, and a transcription terminator. The vector may also contain sequences useful for the amplification of gene expression.

Expression and cloning vectors can and often do contain a selection gene or selection marker. Typically, this gene encodes a protein necessary for the survival or growth of the host cell transformed with the vector. Examples of suitable markers include dihydrofolate reductase (DHFR) or neomycin resistance for eukaryotic cells and tetracycline or ampicillin resistance for E. coli. Selection markers in plants include bleomycin, gentamycin, glyphosate, hygromycin, kanamycin, methotrexate, phleomycin, phosphinotricin, spectinomycin, dtreptomycin, sulfonamide and sulfonylureas resistance (Maliga et al., Methods in Plant Molecular Biology, Cold Spring Harbor Laboratory Press, 1995, p. 39). Additional selectable markers include those that confer herbicide resistance, for example, a mutant EPSPV-synthase, which confers glyphosate resistance (Hinchee et al., BioTechnology 91:915-922, 1998). The selection marker can have its own promoter which promoter can be either a constitutive or an inducible promoter.

The vector may also comprise a reporter gene. Reporter genes greatly enhance the ability to monitor gene expression in a number of biological organisms. In chloroplasts of higher plants, β-glucuronidase (uidA, Staub and Maliga, EMBO J. 12:601-606, 1993), neomycin phosphotransferase (nptII, Caner et al., Mol. Gen. Genet. 241:49-56, 1993), adenosyl-3-adenyltransferase (aadA, Svab and Maliga, Proc. Natl. Acad. Sci., USA 90:913-917, 1993), and the Aequorea victoria GFP (Sidorov et al., Plant J. 19:209-216, 1999) have been used as reporter genes (Heifetz, Biochemie 82:655-666, 2000). Each of these genes has attributes that make them useful reporters of chloroplast gene expression, such as ease of analysis, sensitivity, or the ability to examine expression in situ. Based upon these studies, other heterologous proteins have been expressed in the chloroplasts of higher plants such as Bacillus thuringiensis Cry toxins, conferring resistance to insect herbivores (Kota et al., Proc. Natl. Acad. Sci., USA 96:1840-1845, 1999), or human somatotropin (Staub et al., Nat. Biotechnol. 18:333-338, 2000), a potential biopharmaceutical. Several reporter genes have been expressed in the chloroplast of the eukaryotic green alga, C. reinhardtii, including aadA (Goldschmidt-Clermont, Nucl. Acids Res. 19:4083-4089 1991; Zerges and Rochaix, Mol. Cell. Biol. 14:5268-5277, 1994), uidA (Sakamoto et al., Proc. Natl. Acad. Sci., USA 90:477-501, 1993, Ishikura et al., J. Biosci. Bioeng. 87:307-314 1999), Renilla luciferase (Minko et al., Mol. Gen. Genet. 262:421-425, 1999) and the amino glycoside phosphotransferase from Acinetobacter baumanii, aphA6 (Bateman and Purton, Mol. Gen. Genet. 263:404-410, 2000).

In one embodiment, the proteins encoded by the nucleic acids described herein are modified at the C-terminus by the addition of a Flag-tag epitope to add in the detection of protein expression, and to facilitate protein purification. Affinity tags can be appended to proteins so that they can be purified from their crude biological source using an affinity technique. These include, for example, chitin binding protein (CBP), maltose binding protein (MBP), and glutathione-S-transferase (GST). The poly (His) tag is a widely-used protein tag that binds to metal matrices. Some affinity tags have a dual role as a solubilization agent, such as MBP, and GST. Chromatography tags are used to alter chromatographic properties of the protein to afford different resolution across a particular separation technique. Often, these consist of polyanionic amino acids, such as FLAG-tag. Epitope tags are short peptide sequences which are chosen because high-affinity antibodies can be reliably produced in many different species. These are usually derived from viral genes, which explain their high immunoreactivity. Epitope tags include, but are not limited to, V5-tag, c-myc-tag, and HA-tag. These tags are particularly useful for western blotting and immunoprecipitation experiments, although they also find use in antibody purification. Fluorescence tags are used to give visual readout on a protein. GFP and its variants are the most commonly used fluorescence tags. More advanced applications of GFP include using it as a folding reporter (fluorescent if folded, colorless if not).

In one embodiment, the proteins encoded by the nucleic acids described herein can be fused at the amino-terminus to the carboxy-terminus of a highly expressed protein (fusion partner). These fusion partners may enhance the expression of the gene. Engineered processing sites, for example, protease, proteolytic, or tryptic processing or cleavage sites, can be used to liberate the protein from the fusion partner, allowing for the purification of the intended protein. Examples of fusion partners that can be fused to the gene are a sequence encoding the mammary-associated serum amyloid (M-SAA) protein, a sequence encoding the large and/or small subunit of ribulose bisphosphate carboxylase, a sequence encoding the glutathione S-transferase (GST) gene, a sequence encoding a thioredoxin (TRX) protein, a sequence encoding a maltose-binding protein (MBP), a sequence encoding any one or more of E. coli proteins NusA, NusB, NusG, or NusE, a sequence encoding a ubiqutin (Ub) protein, a sequence encoding a small ubiquitin-related modifier (SUMO) protein, a sequence encoding a cholera toxin B subunit (CTB) protein, a sequence of consecutive histidine residues linked to the 3′ end of a sequence encoding the MBP-encoding malE gene, the promoter and leader sequence of a galactokinase gene, and the leader sequence of the ampicillinase gene.

In some embodiments, the vector, and in particular an expression vector, may comprise nucleotide sequences that are codon-biased for expression in the organism being transformed. In another embodiment, a gene of interest may comprise nucleotide sequences that are codon-biased for expression in the organism being transformed. Alternatively, a gene may comprise nucleotide sequences that are codon-biased for expression in the chloroplast of the organism being transformed.

The skilled artisan is well aware of the “codon-bias” exhibited by a specific host cell in usage of nucleotide codons to specify a given amino acid. Without being bound by theory, by using a host cell's preferred codons, the rate of translation may be greater. Therefore, when synthesizing a gene for improved expression in a host cell, it may be desirable to design the gene such that its frequency of codon usage approaches the frequency of preferred codon usage of the host cell. In some organisms, codon bias differs between the nuclear genome and organelle genomes, thus, codon optimization or biasing may be performed for the target genome (e.g., nuclear codon biased, chloroplast codon biased).

The term “biased,” when used in reference to a codon, means that the sequence of a codon in a polynucleotide has been changed such that the codon is one that is used preferentially in the target which the bias is for, e.g., alga cells, chloroplasts. A polynucleotide that is biased for a particular codon usage can be synthesized de novo, or can be genetically modified using routine recombinant DNA techniques, for example, by a site directed mutagenesis method, to change one or more codons such that they are biased for chloroplast codon usage. Codon bias can be variously skewed in different plants, including, for example, in alga as compared to tobacco. Generally, the codon bias selected reflects codon usage of the plant (or organelle therein) which is being transformed with the nucleic acids of the present invention. For example, where C. reinhardtii is the host, the chloroplast codon usage may be biased to reflect nuclear or chloroplast codon usage (e.g., about 74.6% AT bias in the third codon position for sequences targeting the chloroplast). Preferred codon usage in the chloroplasts of algae has been described in US 2004/0014174.

The basic techniques used for transformation and expression in photosynthetic microorganisms are similar to those commonly used for E. coli, Saccharomyces cerevisiae and other species. Transformation methods customized for a photosynthetic microorganisms, e.g., the chloroplast of a strain of algae, are known in the art. These methods have been described in a number of texts for standard molecular biological manipulation (see Packer & Glaser, 1988, “Cyanobacteria”, Meth. Enzymol., Vol. 167; Weissbach & Weissbach, 1988, “Methods for plant molecular biology,” Academic Press, New York, Sambrook, Fritsch & Maniatis, 1989, “Molecular Cloning: A laboratory manual,” 2nd edition Cold Spring Harbor Laboratory Press, Cold Spring Harbor, N.Y.; and Clark M S, 1997, Plant Molecular Biology, Springer, N.Y.). These methods include, for example, biolistic devices (see, for example, Sanford, Trends In Biotech (1988) δ: 299-302, and U.S. Pat. No. 4,945,050), electroporation (Fromm et al., Proc. Nat'l. Acad. Sci. (USA) (1985) 82: 5824-5828), use of a laser beam, electroporation, microinjection, or any other method capable of introducing DNA into a host cell.

Plastid transformation is a routine and well known method for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome. In some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation (Svab et al., Proc. Natl. Acad. Sci., USA 87:8526-8530, 1990), and can result in stable homoplasmic transformants, at a frequency of approximately one per 100 bombardments of target leaves.

Microprojectile mediated transformation also can be used to introduce a polynucleotide into a plant cell (Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a plant tissue using a device such as the BIOLISTIC PD-1000 particle gun (BioRad; Hercules Calif.). Methods for the transformation using biolistic methods are well known in the art (see, e.g.; Christou, Trends in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (Duan et al., Nature Biotech. 14:494-498, 1996; Shimamoto, Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, the glass bead agitation method, and the like.

A further refinement in chloroplast transformation/expression technology that facilitates control over the timing and tissue pattern of expression of introduced DNA coding sequences in plant plastid genomes has been described in PCT International Publication WO 95/16783 and U.S. Pat. No. 5,576,198. This method involves the introduction into plant cells of constructs for nuclear transformation that provide for the expression of a viral single subunit RNA polymerase and targeting of this polymerase into the plastids via fusion to a plastid transit peptide. Transformation of plastids with DNA constructs comprising a viral single subunit RNA polymerase-specific promoter specific to the RNA polymerase expressed from the nuclear expression constructs operably linked to DNA coding sequences of interest permits control of the plastid expression constructs in a tissue and/or developmental specific manner in plants comprising both the nuclear polymerase construct and the plastid expression constructs. Expression of the nuclear RNA polymerase coding sequence can be placed under the control of either a constitutive promoter, or a tissue- or developmental stage-specific promoter, thereby extending this control to the plastid expression construct responsive to the plastid-targeted, nuclear-encoded viral RNA polymerase.

When nuclear transformation is utilized, the genes encoding the proteins can be modified for plastid targeting by employing plant cell nuclear transformation constructs wherein DNA coding sequences of interest are fused to any of the available transit peptide sequences capable of facilitating transport of the encoded proteins into plant plastids, and driving expression by employing an appropriate promoter. Targeting of the protein can be achieved by fusing DNA encoding plastid, e.g., chloroplast, leucoplast, amyloplast, etc., transit peptide sequences to the 5′ end of DNAs encoding the proteins. The sequences that encode a transit peptide region can be obtained, for example, from plant nuclear-encoded plastid proteins, such as the small subunit (SSU) of ribulose bisphosphate carboxylase, EPSP synthase, plant fatty acid biosynthesis related genes including fatty acyl-ACP thioesterases, acyl carrier protein (ACP), stearoyl-ACP desaturase, β-ketoacyl-ACP synthase and acyl-ACP thioesterase, or LHCPII genes, etc. Plastid transit peptide sequences can also be obtained from nucleic acid sequences encoding carotenoid biosynthetic enzymes, such as GGPP synthase, phytoene synthase, and phytoene desaturase. Other transit peptide sequences are disclosed in Von Heijne et al. (1991) Plant Mol. Biol. Rep. 9: 104; Clark et al. (1989) J. Biol. Chem. 264: 17544; della-Cioppa et al. (1987) Plant Physiol. 84: 965; Romer et al. (1993) Biochem. Biophys. Res. Commun. 196: 1414; and Shah et al. (1986) Science 233: 478. The encoding sequence for a transit peptide effective in transport to plastids can include all or a portion of the encoding sequence for a particular transit peptide, and may also contain portions of the mature protein encoding sequence associated with a particular transit peptide. Numerous examples of transit peptides that can be used to deliver target proteins into plastids exist, and the particular transit peptide encoding sequences useful in the present invention are not critical as long as delivery into a plastid is obtained. Proteolytic processing within the plastid then produces the mature enzyme. This technique has proven successful with enzymes involved in polyhydroxyalkanoate biosynthesis (Nawrath et al. (1994) Proc. Natl. Acad. Sci. USA 91: 12760), and neomycin phosphotransferase II (NPT-II) and CP4 EPSPS (Padgette et al. (1995) Crop Sci. 35: 1451), for example.

Of interest are transit peptide sequences derived from enzymes known to be imported into the leucoplasts of seeds. Examples of enzymes containing useful transit peptides include those related to lipid biosynthesis (e.g., subunits of the plastid-targeted dicot acetyl-CoA carboxylase, biotin carboxylase, biotin carboxyl carrier protein, α-carboxy-transferase, plastid-targeted monocot multifunctional acetyl-CoA carboxylase (Mr, 220,000); plastidic subunits of the fatty acid synthase complex (e.g., acyl carrier protein (ACP), malonyl-ACP synthase, KASI, KASII, KASIII, etc.); steroyl-ACP desaturase; thioesterases (specific for short, medium, and long chain acyl ACP); plastid-targeted acyl transferases (e.g., glycerol-3-phosphate: acyl transferase); enzymes involved in the biosynthesis of aspartate family amino acids; phytoene synthase; gibberellic acid biosynthesis (e.g., ent-kaurene synthases 1 and 2); and carotenoid biosynthesis (e.g., lycopene synthase).

Additional embodiments provide a plastid, and in particular a chloroplast, transformed with a polynucleotide encoding a protein of the present disclosure, for example a chlorophyllase. The protein may be introduced into the genome of the plastid using any of the methods described herein or otherwise known in the art. The plastid may be contained in the organism in which it naturally occurs. Alternatively, the plastid may be an isolated plastid, that is, a plastid that has been removed from the cell in which it normally occurs. Methods for the isolation of plastids are known in the art and can be found, for example, in Maliga et al., Methods in Plant Molecular Biology, Cold Spring Harbor Laboratory Press, 1995; Gupta and Singh, J. Biosci., 21:819 (1996); and Camara et al., Plant Physiol., 73:94 (1983). The isolated plastid transformed with a protein of the present disclosure can be introduced into a host cell. The host cell can be one that naturally contains the plastid or one in which the plastid is not naturally found.

Also within the scope of the present disclosure are artificial plastid genomes, for example chloroplast genomes, that contain nucleotide sequences encoding the proteins of the present disclosure. Methods for the assembly of artificial plastid genomes can be found in co-pending U.S. patent application Ser. Nos. 12/287,230 filed Oct. 6, 2008, published as U.S. Publication No. 2009/0123977 on May 14, 2009, and 12/384,893 filed Apr. 8, 2009, published as U.S. Publication No. 2009/0269816 on Oct. 29, 2009.

In some instances, the composition produced by a modified photosynthetic organism is a combination of two or more, 3 or more, 4 or more, 5 or more, 6 or more, 7 or more, 8 or more, 9 or more, 10 or more, 15 or more, or 20 or more different chemical species, such as in the case of a fuel product. Such a fuel product can resemble crude oil (e.g., a mix of hydrocarbons) or in some embodiments, a mixture of fatty acids or lipids, and hydrocarbons resembling gasoline or any other combustible fuel.

The product produced by a photosynthetic organism can be purified in whole, or in part. Thus, one embodiment contemplates obtaining an organism (e.g. a genetically modified NVPO) or biomass, and removing some (e.g. greater than about 30, about 40, about 50, about 60, about 70, about 80, about 90 or about 99%) or all of the product. An organism producing a product, such as a modified organism, can have a product that is further modified to remove the nitrogen from the chlorophyll and/or pheophytin in the product.

Nitrogen Removal from a Chlorophyll Containing Product

Nitrogen oxides (i.e. gases containing nitrogen and oxygen that include NO, NO₂, NO₃, N₂O, N₂O₃, N₂O₄, and N₂O₅ which are here after referred to as NOₓ) can be produced from the combustion of fuels containing nitrogen and can be harmful to the environment, including plants, animals and humans. Therefore, the amount of NOₓ emissions resulting from the combustion of certain fuels (e.g. from factories and combustion powered vehicles) is tightly regulated and monitored in most countries. High NOₓ emissions can be correlated to the nitrogen content of a fuel. Biofuel products obtained from photosynthetic organism can contain high amounts of nitrogen. This can be due to nitrogen containing pigments (e.g. chlorophyll and/or pheophytin) that contaminate a refined fuel product. For example, wherein a chlorophyll containing product is a biofuel, removal of nitrogen containing chlorophyll can lower NOₓ emissions resulting from combustion of such fuels.

Chlorophyll

Several different forms of chlorophyll exist in photosynthetic organisms. Non-limiting examples of chlorophyll molecules include Chlorophyll a, Chlorophyll b, Chlorophyll c, Chlorophyll c1, Chlorophyll c2, Chlorophyll d, Pheophytin a, Pheophytin b, Chlorovitamin K1, Chlorophyllins, Chlorophyllin b, Methylpheophorbide, Bacteriochlorophyll d, Chlorophyllypt, Methylchlorophyllide A, Methylchlorophyllide B, Chlorophyllide b, Chlorophyllide a, Protochlorophyll, Pheophorbide a, Pheophorbide b, Bacteriopheophytin, Bacteriochlorophyll, 4-Divinylprotochlorophyllide, Pheophorbide b, Protochlorophyllide, Zinc chlorophyll b, Chlorophyll a′, 4-Vinylprotochlorophyll a, Pyropheophorbide a, Copper chlorophyll sodium, Copper chlorophyllin A, Phylloerythrin, Sodium copper hlorophyllin, Methyl phaeoporphyrin a, Sodium iron chlorophyllin, Cobalt chlorophyllin, Chlorophyll P 700, Chlorophyll P 680, Bacterioviridin, Methyl bacteriopheophorbide c, Methylpheophorbide-a-(hexyl-ether), Monovinyl protochlorophyllide b, and 2-(1-Hexyloxyethyl)-2-devinyl pyropheophorbide-a.

As used herein, the term chlorophyll or chlorophyll-like molecule refers to any and all forms of chlorophyll and to any and all molecules found in photosynthetic organisms that comprise a porphyrin ring structure similar to that found in chlorophyll. These two terms can be used interchangeably throughout the specification. The nitrogen content of chlorophyll can be found in the porphyrin ring structure. All forms of chlorophyll consist of a porphyrin head, referred to as the porphyrin ring, formed by four linked pyrrole rings with a divalent cation (e.g. magnesium) chelated at the center by four nitrogen atoms. Porphyrin rings can differ slightly among different forms of chlorophyll and a porphyrin ring can have several different side chains. The porphyrin ring of some chlorophyll forms (e.g. chlorophyll a, b and d) is covalently bonded to a hydrophobic tail, sometimes referred to as the phytol tail. The phytol tail adds to the hydrophobic properties of chlorophyll which can be responsible for its ability to contaminate certain products (e.g. biofuel products) during a refining process.

Product Isolation

As disclosed herein, the product isolation process can comprises partial or complete removal of nitrogen or a porphyrin ring from a biofuel product. For example, this can be accomplished by removal of a hydrophobic side chain (e.g. phytol) from the porphyrin ring of a chlorophyll or a chlorophyll-like molecule and/or a pheophytin. After removal of the hydrophobic side chain, the porphyrin ring may become less soluble in an organic solvent. A compound with such a porphyrin ring may precipitate or become more readily adsorbed onto an adsorbent. In other instances, after removal of a hydrophobic side chain, the porphyrin ring can become more soluble in an aqueous or polar solvent and can be removed from the product or biomass by any suitable method (e.g. by an extraction or washing). For example a method for producing a refined product (e.g. a nitrogen-depleted product) can comprise degrading a chlorophyll and/or a pheophytin in a composition comprising chlorophyll and/or a pheophytin and a product, removing from the composition a cleaved portion of the chlorophyll and/or a pheophytin comprising nitrogen, and refining the remainder of the composition to produce a refined product (e.g. nitrogen-depleted product). The degrading step can be complete or partial such that all or substantially all of the nitrogen is removed from the refined product. In some aspects a product that is substantially free of nitrogen contains up to about 0.1% nitrogen (w/w). In some aspects, a product that is substantially free of nitrogen contains up to 0.08%, 0.06%, 0.04%, 0.02%, 0.01%, 0.008%, 0.006%, 0.004%, or 0.002% nitrogen (w/w). In one aspect, the product comprises one or more isoprenoids. In one example, the isoprenoid is a monoterpene, sesquiterpene, diterpene, sesterpene, triterpene, carotenoid, squalene or neophytadiene. Removal or separation of a porphyrin ring structure from a product (e.g. a biofuel) can result in a reduction in the nitrogen content of the final product. When the final product is a fuel, this can result in reduced NOₓ emissions upon combustion of said fuel.

A photosynthetic organism can be prepared for removal of nitrogen using any method known in the art. Non-limiting examples include, harvesting the algae or concentrating the algae by removing the water. The starting material can be any biomass, wet or dry or semi-dry, comprising chlorophyll and/or a pheophytin and a product or interest (e.g. lipids such as fatty acids, or hydrocarbons). A product-containing biomass can be derived from culturing or fermentation of a photosynthetic organism.

The photosynthetic organism can be prokaryotic or eukaryotic. In some cases, the photosynthetic organism can be non-vascular. In other cases, the photosynthetic organism can be photosynthetic and vascular. The photosynthetic organism can be eukaryotic or prokaryotic. The photosynthetic organism can be unicellular or multicellular. The photosynthetic organism can be one that naturally photosynthesizes (has a plastid) or that is genetically engineered or otherwise modified to be photosynthetic. In some instances, the photosynthetic organism can be transformed with a nucleic acid which renders all or part of the photosynthetic apparatus inoperable.

Examples of some prokaryotic organisms of the present disclosure include, but are not limited to, cyanobacteria (e.g., Synechococcus, Synechocystis, Athrospira, Gleocapsa, Oscillatoria, and Pseudoanabaena). In some embodiments, the host organism is a eukaryotic algae (e.g. green algae, red algae, or brown algae). In some embodiments the algae is a green algae, for example a Chlorophycean. The algae can be unicellular or multicellular algae. In some instances the organism is a rhodophyte, chlorophyte, heterokontophyte, tribophyte, glaucophyte, chlorarachniophyte, euglenoid, haptophyte, cryptomonad, dinoflagellum, or phytoplankton. In other embodiments, the host cell is a microalga (e.g., Chlamydomonas reinhardtii, Dunaliella sallna, Haematococcus pluvialis, Scenedesmus dimorphus, Chlorella spp., D. viridis, or D. tertiolecta).

In a one embodiment, the photosynthetic organism is a plant. The term “plant” is used broadly herein to refer to a eukaryotic organism containing plastids, particularly chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Exemplary useful parts of a plant include harvestable parts and parts useful for propagation of progeny plants. A harvestable part of a plant can be any useful part of a plant, for example, flowers, pollen, seedlings, tubers, leaves, stems, fruit, seeds, roots, and the like. A part of a plant useful for propagation includes, for example, are seeds, fruits, cuttings, seedlings, tubers, rootstocks, and the like.

In other embodiments the photosynthetic organism is a vascular plant. Non-limiting examples of such plants include various monocots and dicots, including high oil seed plants such as high oil seed Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oilnut trees such as olive (Olea europaea), sesame, and peanut (Arachis hypogaea), as well as Arabidopsis, tobacco, wheat, barley, oats, amaranth, potato, rice, tomato, and legumes (e.g., peas, beans, lentils, alfalfa, etc.).

Non-limiting examples of non-vascular photosynthetic microorganisms include algae (e.g. red algae, green algae), protists (such as euglena) and bacteria (such as cyanobacteria). In one aspect, the algae is Chlamydomonas, Scenedesmus, Chlorella or Nannochlorpis. In another aspect, the algae is C. reinhardtii. In yet another aspect, the algae is C. reinhardtii 137c. Additional non-limiting examples of non-vascular photosynthetic organisms include cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustigmatophyta, raphidophyta, phaeophyta, and phytoplankton.

Some of the photosynthetic organisms which may be used are halophilic (e.g., Dunaliella salina, D. viridis, or D. tertiolecta). For example, D. salina can grow in ocean water and salt lakes (salinity from about 30-about 300 parts per thousand) and high salinity media (e.g., artificial seawater medium, seawater nutrient agar, brackish water medium, seawater medium, etc.). In some embodiments, a photosynthetic organism for use in the present disclosure can be grown in a liquid environment which is about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 31., about 3.2, about 3.3, about 3.4, about 3.5, about 3.6, about 3.7, about 3.8, about 3.9, about 4.0, about 4.1, about 4.2, or about 4.3 molar or higher concentrations of sodium chloride. One of skill in the art will recognize that other salts (sodium salts, calcium salts, potassium salts, etc.) may also be present in the liquid environments.

When a halophilic organism is utilized, it may be transformed with any of the methods described herein. For example, D. salina may be transformed with a vector which is capable of insertion into the chloroplast genome and which contains nucleic acids which encode a protein disclosed herein. Transformed halophilic organisms may then be grown in high-saline environments (e.g., salt lakes, salt ponds, high-saline media, etc.).

A host algae transformed to produce a protein described herein, for example, a chlorophyllase, can be grown on land, e.g., ponds, aqueducts, landfills, or in closed or partially closed bioreactor systems. Algae can also be grown directly in water, e.g., in oceans, seas, on lakes, rivers, reservoirs, etc. In embodiments where algae are mass-cultured, the algae can be grown in high density photobioreactors. Methods of mass-culturing algae are known in the art. For example, algae can be grown in high density photobioreactors (see, e.g., Lee et al, Biotech. Bioengineering 44:1161-1167, 1994) and other bioreactors (such as those for sewage and waste water treatments) (e.g., Sawayama et al, Appl. Micro. Biotech., 41:729-731, 1994). Additionally, algae may be mass-cultured to remove heavy metals (e.g., Wilkinson, Biotech. Letters, 11:861-864, 1989), hydrogen (e.g., U.S. Patent Application Publication No. 20030162273), and pharmaceutical compounds.

The photosynthetic organism (e.g. genetically modified algae) can be grown under any suitable condition, for example under conditions which permit photosynthesis or in the absence of light.

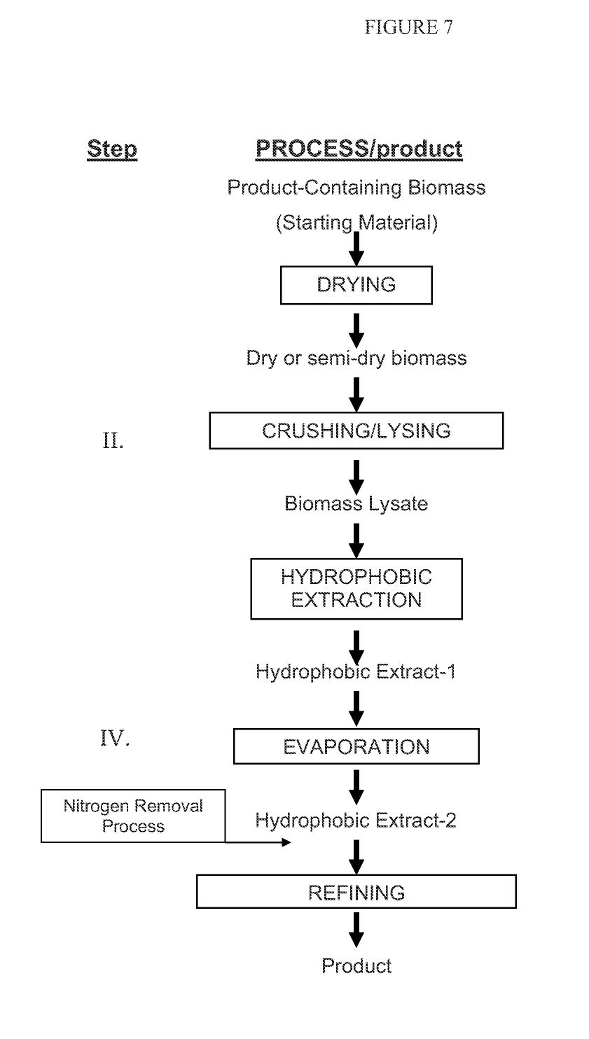

The product-containing biomass can be harvested from its growth environment (e.g. lake, pond, photobioreactor, or partially closed bioreactor system, etc.) using any suitable method. Non-limiting examples of harvesting techniques are centrifugation or flocculation. Once harvested, the product-containing biomass can be subjected to a drying process (for example, as shown in step I of FIG. 7). Alternately, an extraction step may be performed on wet biomass. The product-containing biomass can be dried using any suitable method. Non-limiting examples of drying methods include sunlight, rotary dryers, flash dryers, vacuum dryers, ovens, freeze dryers, hot air dryers, microwave dryers and superheated steam dryers. After the drying process the product-containing biomass can be referred to as a dry or semi-dry biomass. The moisture content of the dry or semi-dry biomass can be up to about 20%, about 15%, about 10%, about 5%, about 4%, about 3%, about 2% or about 1% (wt/wt).

A dry or semi-dry biomass can be further subjected to a crushing/lysis/pelletizing/milling/extrusion/flaking step (for example, as shown in FIG. 7, Step II) by any suitable method. This step is optional. The crushing/lysis step comprises physical, chemical or enzymatic disruption of the cells in the dry or semi-dry biomass. Physical disruption may be accomplished by any suitable method including but not limited to shredding, grinding, crushing, rolling, flaking, sonication or variations of the French press technology. In some instances the dry or semi-dry biomass is subjected to crushing/lysis/pelletizing/milling/extrusion/flaking by means of a roller (flaker) device. Chemical disruption may be accomplished by using any chemical compound or mixture that breaks down the structural constituents of a cell and/or lyses the cells. Such chemicals may include a variety of acids, (e.g hydrochloric acid, nitric acid, acetic acid, sulfuric acid, or phosphoric acid), bases (e.g. bleach, sodium hydroxide, potassium hydroxide, sodium carbonate, calcium carbonate, calcium hydroxide, solid catalysts, or ammonia), detergents, and hypotonic or hypertonic solutions. Enzymatic disruption may be accomplished by using any enzyme or enzyme mixture that breaks down the structural constituents of a cell and/or lyses the cell. Any one of the above described disruption processes can optionally be used in conjunction with one or more of the other processes of disruption. Further, when a dry or semi-dry biomass is subjected to more than one process of disruption, such processes may occur simultaneously or in a step-wise fashion. After the crushing/lysis step is complete, a biomass lysate suitable for extraction (for example, as shown in FIG. 7, Step III) can be produced.

The biomass lysate can be extracted (for example, as shown in FIG. 7, Step III) using any suitable method (e.g. solvent based extraction) and may utilize any solvent miscible or, immiscible with water, such as alcohols, hydrocarbon solvents, aromatic solvents, acetone, glycerol, alcohol, hexane, heptane, methylpentane, toluene, or methylisobutylketone. If the solvent is an alcohol, examples of alchohols include, but are not limited to, methanol, propanol, ethanol, and isopropanol. In one example, a method for producing a refined product can comprise an extraction step. The hydrophobic extraction process results in an extract (e.g. extract-1, FIG. 7) that can be rich in lipids, fatty acids, and hydrocarbons. When the product of interest is a biofuel, the product can be found in extract-1. The solvent used for the hydrophobic extraction can be immiscible with water. Solvents which partition between water and organic solvents to leave a major part of the solvent in the water phase are also contemplated for the hydrophobic extraction step. Non-limiting examples of solvents suitable for the hydrophobic extraction step include non-polar organic liquids, especially aliphatic hydrocarbons, such as hexane or various petroleum ethers. Other non-limiting examples of solvents include esters, ethers, ketones, and nitrated and chlorinated hydrocarbons. Additional non-limiting examples of water-immiscible solvents contemplated for use in the hydrophobic extraction step include carbon tetrachloride, chloroform, cyclohexane, 1,2-dichloroethane, dichloromethane, diethyl ether, dimethyl formamide, ethyl acetate, heptane, hexane, methyl-tert-butyl ether, pentane, toluene, or 2,2,4-trimethylpentane. In one non-limiting example, a biomass lysate is subjected to extraction with hexane. One embodiment contemplates using a combination of two or more solvents either together or in series. Thus, in one example, a biomass lysate can be extracted using mixtures of solvents that include aliphatic or acyl alcohols.

Hydrophobic extraction of the biomass lysate can utilize any suitable extraction method. Extractors can be operated in crosscurrent or counter-current mode. Crosscurrent mode is mostly used in batch operation. Batch extractors have traditionally been used in low capacity multi-product plants such as are typical in the pharmaceutical and agrochemical industries. For washing and neutralization operations that require very few stages, crosscurrent operation can be particularly practical and economical and offers a great deal of flexibility. Crosscurrent extraction devices can comprise an agitated tank that can also be used for other reaction steps (e.g. chlorophyll hydrolysis). In these tanks, solvent is first added to the biomass lysate, the contents are mixed, settled and then separated. Single stage extraction can be used when the extraction is fairly simple and can be achieved without a high amount of solvent. If more than one stage is required, multiple solvent-washes can be performed. For larger volume operation and more efficient use of solvent, countercurrent column extractors can be employed. Countercurrent column extractors can be static or agitated. Non-limiting examples of countercurrent static column extractors include spray columns, sieve columns and packed columns. Non-limiting examples of agitated columns include rotating disc contactors, scheibel columns, Kuhni columns, Karr columns and pulsed columns. In one non-limiting example, a biomass lysate is extracted using hexane and any suitable countercurrent extractor. Extraction may also be performed using a wet bead mill.

Following the hydrophobic extraction step, extract-1 can contain solvent that requires removal. A solvent may be removed after or before the removal of chlorophyll. Solvent can be removed by any suitable method, for example, evaporation. In one non-limiting example, solvent (e.g. hexane) can be removed by evaporation. Therefore, in one example, a method for producing a refined product can comprise an evaporation step. In some aspects, extract-1 can be subjected to an evaporation step to remove solvent (e.g. hexane) resulting in extract-2 (see e.g. FIG. 7, Step IV).

In the final step of the exemplary process described above (FIG. 7), extract-2 can be further subjected to a refining process to produce the product (e.g. a final product). Therefore, when the product is a biofuel, a refining process can comprise a cracking step. A cracking step can be used to convert an extract (e.g. extract-2) to a useable liquid hydrocarbon fuel (e.g. biofuel) using any suitable cracking technology (e.g., catalytic cracking or use of an alkaline catalyst). Many such methodologies are known in the art including, but not limited to, cracking, hydrogenation, fractionation, distillation, catalytic cracking, direct pyrolysis, use of alkaline catalyst, hydrolysis, decarboxylation, dehydration, hydrothermolysis (U.S. application Ser. No. 11/857,937), isomerization, cyclization, aromatization, acid-catalyzed pretreatment followed by alkaline catalyzed transesterification, thermal cracking, use of equilibrium catalysts and fluid catalytic cracking (See, e.g., Dupain et al., Applied Catalysis B: Environmental 72(1-2): 44-61 (2007); Ooi et al., Biomass and Bioenergy 27(5):477-84 (2004); Bhatia, S., Reaction Kinetics and Catalysis Letters, 84(2); 295-302 (2005); Canakci, et al., Transactions of the ASAE, 46(4); 945-54 (2003); PCT Pub. No. WO2007/068097; U.S. Pat. No. 7,288,685). In one example, a method for producing a refined product (e.g. a nitrogen-depleted product) can comprise degrading a chlorophyll and/or a pheophytin in a composition comprising chlorophyll and/or a pheophytin and a product, removing from the composition a cleaved portion of the chlorophyll and/or a pheophytin comprising nitrogen, and refining the remainder of the composition to produce the product (e.g. nitrogen-depleted product). In one aspect, refining of the remaining composition can comprise cracking.

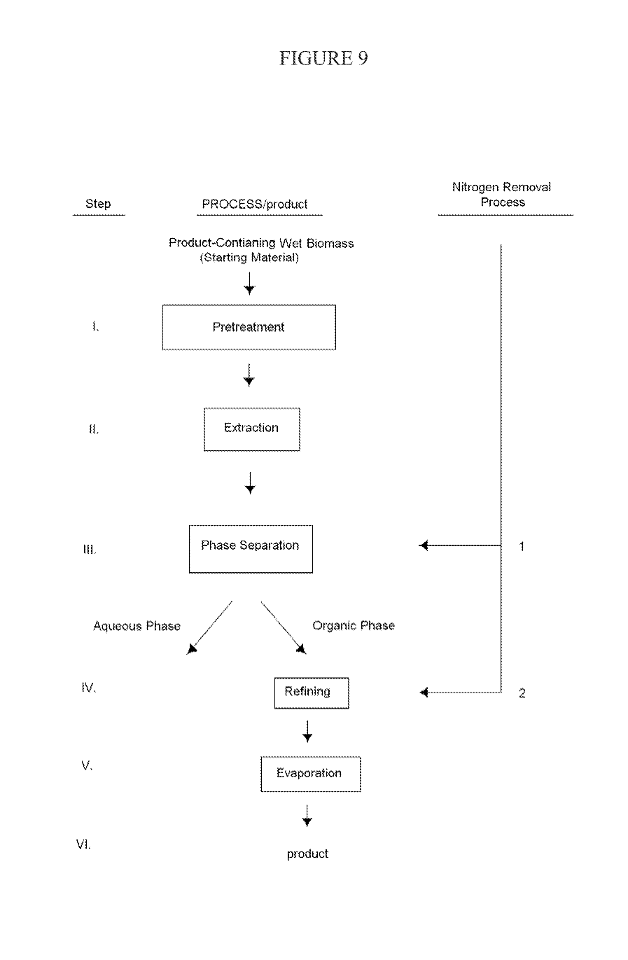

FIG. 9 is a flow diagram illustrating an exemplary method of integrating a nitrogen removal process into a refining method using a product-containing wet biomass as a starting material. The refining and evaporation steps can be repeated as many times as needed. Optionally, an additional refining step can be added after the refining and evaporation step. This method can be used to produce a nitrogen-depleted product from a photosynthetic organism. Nitrogen can be removed during several steps of the overall process as shown by the arrows.

The starting material is a product-containing wet biomass or biomass composition. In some embodiments, the product-containing biomass or biomass composition is obtained from a naturally occurring non-vascular photosynthetic organism (e.g. algae). In another embodiment, the product-containing biomass or biomass composition is obtained from a genetically modified non-vascular photosynthetic organism (e.g. algae). The biomasss composition comprises one or more chlorophylls and/or a pheophytins and a product of interest. For example, the product of interest can be a lipid, such as an oil.

The biomass composition is then pretreated to degrade at least a subset of the chlorophyll and/or pheophytin in the biomass composition. The breakage of the ester bonds may occur during the pretreatment, the extraction, or the refining step. During the pretreatment step in FIG. 9, ester bonds, such as those found in lipids are cleaved. For example, lipids are cleaved into a free fatty acid and a alcohol, a sterol, a glycerol, or a polar group. The polar group can contain a phosphate, a sulfur, or a sugar group, for example. Chlorophyll can be cleaved into phytol and chlorophyllide. Phytol can be further converted into phytadiene. Pheophytin can be cleaved into phytol and pheophorbide. Phytol can be further converted into phytadiene. A triglyceride can be cleaved into a free fatty acid and glycerol. The pretreatment step in FIG. 9 can be an enzyme treatment (addition of a chlorophyllase, for example), an acid treatment, a base treatment, a low temperature treatment, or a high temperature treatment, for example.

A low temperature treatment consists of cooling the biomass composition to about 0 degrees Celsius to about −40 degrees Celsius, about −5 degrees Celsius to about −20 degrees Celsius, or to about −20 degrees Celsius, for example.

A high temperature treatment consists of heating the biomass composition to about 60 degrees Celsius to about 250 degrees Celsius, about 80 degrees Celsius to about 200 degrees Celsius, or to about 120 degrees Celsius, for example. High temperature treatments may include the addition of glycerol. For example, a ratio of glycerol to ash free dry weight (AFDW) biomass of 10 to 1 by mass are heated at 200° C. with a catalyst.

Chemicals that may be used in the pretreatment step may include any variety of acids, (e.g hydrochloric acid, nitric acid, acetic acid, sulfuric acid, or phosphoric acid), bases (e.g. bleach, sodium hydroxide, potassium hydroxide, solid catalysts, ammonia, sodium carbonate, calcium carbonate, or calcium hydroxide), detergents, and hypotonic or hypertonic solutions.

The pretreatment or degrading step can comprise the use of one or more acids. The acid may be an organic acid or inorganic acid. Other embodiments include where the acid is hydrochloric acid, citric acid, nitric acid, acetic acid, sulfuric acid, formic acid, phosphoric acid, succinic acid, or a solid acid catalyst. Exemplary solid acid catalysts that can be used are an acidified aluminum oxide, acidified silicon dioxide, acidified zironium hydroxide, acidified zeolite, or activated carbon.

The pretreatment or degrading step can comprise the use of one or more bases. Exemplary bases are bleach, sodium hydroxide, potassium hydroxide, ammonia, sodium carbonate, calcium carbonate, calcium hydroxide, or a solid base catalyst. Exemplary solid base catalysts that can be used are calcium methoxide, calcium oxide, potassium hydroxide/aluminum oxide, or magnesium oxide.

The pretreatment step may include enzymatic disruption that is accomplished by using any enzyme or enzyme mixture that breaks down the structural constituents of a cell and/or lyses the cell.

The pretreatment step can include cell disruption by enzymes or mechanical disruption. Non-limiting examples of mechanical disruption are beads, homogenization, shock waves, ultrasound, electromagnetic fields, cavitation mixers, high shear refiners, and electromagnetic pulse.

Any one of the above-described treatments can optionally be used in conjunction with one or more of the other treatments. In addition, treatments may occur simultaneously or in a step-wise fashion. For example, a wet biomass solution can be pretreated with KOH (1 molar) at 60° C. for 1 hour, and then the solution can be brought to pH 2 with concentrated sulfuric acid.

During an extraction step and a phase separation step, the cleaved portion of the degraded chlorophyll or pheophytin comprising nitrogen or nitrogen containing pigments, is removed. The chlorophyllide and/or pheophorbide is removed by dissolving or dispersing the biomass composition in one or more solvents. This cleaved portion, containing the nitrogen can then be found in the aqueous phase.

During the extraction step, one or more solvents are added so that an aqueous layer or phase and an organic layer or phase are formed. Examples of solvents that can be used are water, acetone, glycerol, alcohol, hexane, heptane, methylpentane, toluene, methylisobutylketone, or any two or more of the above. If the solvent is an alcohol, examples of alchohols include, but are not limited to, methanol, propanol, ethanol, and isopropanol.

Extraction can be performed by bead mill, mixed tank, circulated tank, cavitation mixer, ultrasound, homogenizer, high-sheer mixer, shockwave, electromagnetic field, or pressure shock.

The phase separation step is performed by decanting or centrifuging the mixture so that the aqueous phase is separated from the organic phase. The organic phase can contain the solvent(s) and the lipid(s). For example, the emulsion from the bead mill can be centrifuged to separate the aqueous phase containing the extracted biomass (e.g. containing the nitrogen ring from chlorophyll) from the organic phase containing the product (e.g. oil or phytadiene) and the solvent.

The organic phase can be further refined. For example, residual phosphorus, trace metals, trace heteroatoms, residual nitrogen, polar compounds, glycerol backbones, or fatty acids can be removed in the refining step. The unwanted compounds can be removed by absorbance to an absorbant material, for example, bleaching clay or a carbonaceous material. The unwanted compounds can also be removed, for example, by water washing, or by an acid and water wash.

The evaporation step can remove whatever solvents are present in the mixture. Evaporation can be performed using standard equipment and temperature ranges known to one of skill in the art.

Optionally, a further refining step can occur. For example, phosphorus, trace metals, trace heteroatoms, and/or residual nitrogen can be removed in the refining step. Free fatty acids can also be removed by evaporation at high temperatures, deoderization, distillation, steam-stripping, or stream distillation, for example.

After the evaporation step or optional refining step, the desired product or products are obtained. Examples of desired products are neutral lipids, for example, fatty acids, carotenoids, fatty alcohols, sterols, triglycerides, wax esters, and sterol esters. Other examples of desired products are fatty acids, lipids or hydrocarbons. The product can contain one or more hydrocarbons, or one or more isoprenoids. Non-limiting examples of isoprenoids are, monoterpene, sesquiterpene, diterpene, sesterpene, triterpene, carotenoid, squalene or neophytadiene. The product can comprise phytol, phytadiene, or neophytadiene. The product can be naturally found in the photosynthetic organism or not naturally found in the photosynthetic organism.

Examples of compositions that can be obtained from the disclosed methods are: a composition comprising up to about 75% (w/w) free fatty acids and up to about 10% (w/w) phytol; a composition comprising up to about 50% (w/w) free fatty acids and up to about 10% (w/w) phytol; and a composition comprising up to about 85% (w/w) free fatty acids and up to about 10% (w/w) phytol. The remaining % (w/w) up to 100% can be made up, for example, of sterols, carotenoids, hydrocarbons, and neutral lipids.

Other exemplary compositions that can be obtained from the disclosed methods are: a composition comprising up to about 25% (w/w) free fatty acids, up to about 25% triglycerids, and up to about 15% (w/w) wax esters; and a composition comprising up to about 40% (w/w) free fatty acids, up to about 40% triglycerids, and up to about 40% (w/w) wax esters, wherein the total % (w/w) cannot exceed 100%. The remaining % (w/w) up to 100% can be made up, for example, of sterols, steryl esters, carotenoids, glycolipids, and hydrocarbons.

Optionally, the nitrogen-depleted product can comprise about 5% to about 95% free fatty acids (w/w), about 10% to about 90% free fatty acids (w/w), about 50% to about 85% free fatty acids (w/w), or about 85% free fatty acids (w/w), for example. Optionally, heteroatoms can be removed from the nitrogen-depleted product. Examples of heteroatoms that can be removed are one or more of oxygen, phosphorus, nitrogen, sulfur, or metals.

Optionally, fresh solvent can be added back to the solution containing the aqueous phase and extracted biomass, for a second extraction, followed by another phase separation step with the centrifuge, for example, and further refining of the organic phase, including an additional evaporation step, as needed, to obtain the desired product. This process can be done, for example, as a commercial process in continuous counter-current mode. This process can also be done in a cross-current mode.

Optionally, the miscella (solvent and lipid) can be added to the biomass and water, or the wet biomass, for a second extraction, followed by another phase separation step with the centrifuge, for example, and further refining of the organic phase, including an additional evaporation step, as needed, to obtain the desired product. This process can be done as a commercial process in continuous counter-current fashion.

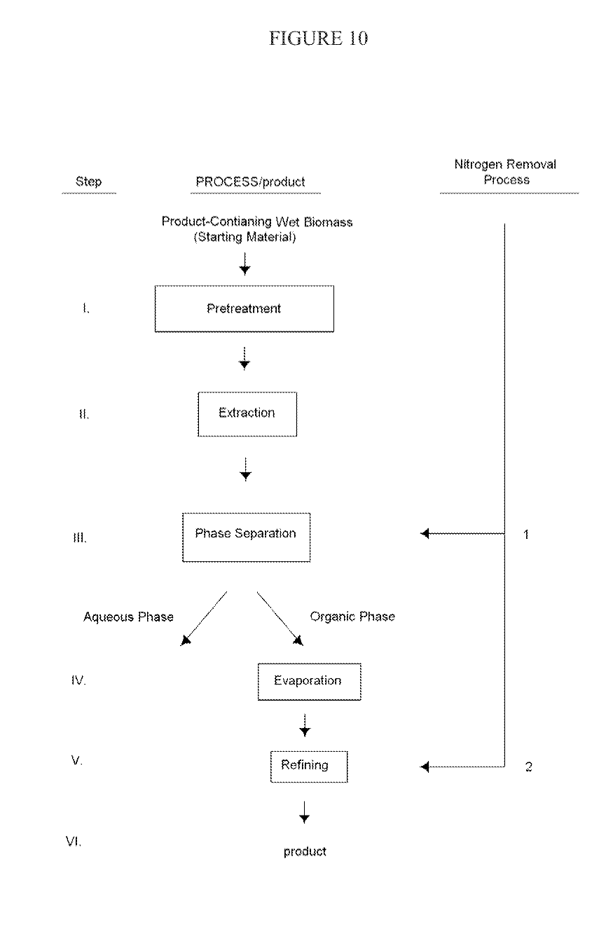

FIG. 10 is flow diagram illustrating another exemplary method of integrating a nitrogen removal process into a refining method using a product-containing wet biomass as a starting material. The refining and evaporation steps are reversed to that shown in FIG. 9, as described above. The evaporation and refining steps can be repeated as many times as needed. This method can be used to produce a nitrogen-depleted product from a photosynthetic organism. Nitrogen can be removed during several steps of the overall process as shown by the arrows.

Other Process Steps

A process of producing a refined product can comprise additional steps, substituted steps or less steps than discussed herein (for example, as shown in FIGS. 7-10). Non-limiting examples of other steps that can be used in producing a refined product include heating, cooling, mixing, holding, hydrating, washing, extracting, filtering, drying, distillation, bleaching, deodorization, decanting, fractionation, separating, phase separation, sediment removal by any means, and centrifugation. Therefore, a method for producing a refined product can comprise degrading a chlorophyll and/or pheophytin, removing a cleaved portion of the chlorophyll and/or pheophytin from the composition, recovering the remaining composition and refining the recovered composition. Alternatively, the chlorophyll and/or pheophytin may be removed with the hydrocarbon tail attached. In such instances, the chlorophyll and/or pheophytin may be subsequently treated to recover the hydrocarbon tail. The refining step can comprise drying, crushing/lysis, extraction, evaporation, cracking, heating, cooling, mixing, holding, hydrating, washing, extracting, filtering, drying, distillation, bleaching, deodorization, deguming, decanting, fractionation, separating, phase separation, sediment removal by any means, or centrifugation.

Wherein the product is a biofuel, a degumming step can be included in the refining process. Degumming is the process of degrading, removing, or partially removing phospholipids (i.e. gums) from a lipid based extract (e.g. extract-2). Phospholipids can have undesirable effects on the further processing of a biofuel. Therefore, in some aspects the compositions and methods as disclosed herein can be used with (i.e., in conjunction with) any “degumming” procedure, including water degumming, ALCON oil degumming (e.g., for soybeans), safinco degumming, “super degumming,” UF degumming, TOP degumming, uni-degumming, dry degumming, and ENZYMAX™ degumming. Examples of degumming processes are described in U.S. Pat. Nos. 6,355,693, 6,162,623, 6,103,505, 6,001,640, 5,558,781, and 5,264,367. Compositions and methods as disclosed herein can be used in any oil processing method, e.g., degumming or equivalent processes. For example, compositions and methods as disclosed herein can be used in processes as described in U.S. Pat. Nos. 5,558,781, 5,288,619, 5,264,367, 6,001,640, 6,376,689, WO 02/29022, oil degumming as described, e.g., in WO 98/18912, processes as described in JP Application No.: H5-132283 (filed Apr. 25, 1993), and EP Application number: 82870032.8. Various “degumming” procedures incorporated by the methods as disclosed herein are described in Bockisch, M. (1998) In Fats and Oils Handbook, The extraction of Vegetable Oils (Chapter 5), 345-445, AOCS Press, Champaign, Ill. The compositions and methods as disclosed herein can be used in the industrial application of enzymatic degumming of triglyceride oils as described, e.g., in EP 513 709. The compositions and methods disclosed herein can be used in the industrial application of enzymatic degumming as described, e.g., in CA 1102795, which describes a method of isolating polar lipids from cereal lipids by the addition of at least 50% by weight of water. This method is a modified degumming in the sense that it utilizes the principle of adding water to a crude oil mixture.

Removal of Nitrogen

Nitrogen removal from a chlorophyll and/or pheophytin containing biomass can comprise two steps, a chlorophyll and/or pheophytin degradation step and a nitrogen extraction step. Nitrogen removal can take place before, after or during any or all steps of the refining process (for example, as shown in FIGS. 7-10). One non-limiting example of integration of a chlorophyll and/or pheophytin degradation step into a refining process is illustrated in FIG. 7. In some instances, chlorophyll and/or pheophytin may be removed from the miscella in the presence of an organic solvent and lipids.

Chlorophyll Degradation

Chlorophyll degradation is a primary biochemical event in nature and results in color changes of photosynthetic organisms. The chlorophyll degradation pathway is described in (Matile P, et. al (1999) Ann. Rev. Plant Physiol. 50: 67-95; Tsuchiya et al., (1999) Proc Natl Acad Sci USA 96: 15362-1 5367; and Benedetti and Arruda (2002) Plant Physiology 128: 1255-1263). Chlorophyllase (e.g. EC 3.1.1.14), one of the major enzymes involved in the first step of chlorophyll degradation, removes the hydrophobic, twenty carbon phytol tail from chlorophyll or a pheophytin (i.e. chlorophyll without a central magnesium ion). Chlorophyll without the phytol tail becomes the light green molecule, chlorophyllide. A pheophytin without the phytol tail becomes a pheophorbide. The lack of the phytol tail can change the solubility. Chlorophyllide and pheophorbide can be soluble in aqueous solutions whereas chlorophyll and pheophytin can be soluble in organic solvents. A chlorophyll or chlorophyllide molecule can be converted to pheophytin or pheophorbide, respectively, by removal of the center chelated divalent magnesium cation. Removal of magnesium can be accomplished by the enzyme magnesium dechelatase (Matile P, et. al (1999) Ann. Rev. Plant Physiol. 50: 67-95; Takamiya, et al. (2000) Trends. Plant. Sci. 5(10):426-431). A pheophorbide can be converted to a red-colored compound, red chlorophyll catabolite (RCC), for example, by the action of the enzyme pheophorbide a oxygenase (Hortensteiner et al., (1998) J Biol Chem 273: 15335-15339; Thomas et al., (2002) J Exp Bot 53: 801-808). An enzyme known as RCC reductase can convert RCC to a fluorescent chlorophyll catabolite (FCC). Other various enzymes can convert FCC to nonfluorescent chlorophyll catabolites.

Any enzyme known in the art can be used to degrade chlorophyll or pheophytin. For example various chlorophyllases have been purified, cloned, and recombinantly expressed from photosynthetic organisms (U.S. Pat. No. 7,199,284) and any given one can be used in the compositions or methods as disclosed herein. Exemplary biomass degrading enzymes, that may be used in the methods described herein are described in International Patent Application No. PCT/US2008/006879, filed May 30, 2008. Additional polypeptides and/or peptides, either recombinantly produced or produced and purified from natural sources, can have esterase activity similar to a chlorophyllase. These polypeptides and/or peptides can include catalytic antibodies, enzymes, and active sites of enzymes. Any chlorophyllase, chlase, or chlorophyll-chlorophyllido hydrolyase or polypeptide having a similar activity (e.g., chlorophyll-chlorophyllido hydrolase 1 or chlase 1, or, chlorophyll-chlorophyllido hydrolase 2 or chlase 2 (e.g., NCBI P59677-1 and P59678, respectively) can be used in a composition or method as disclosed herein. Any polypeptide (e.g., enzyme or catalytic antibody) that catalyses the hydrolysis of a chlorophyll or pheophytin ester bond to yield chlorophyllide and a phytol or pheophorbide and a phytol can be used in a composition or method as disclosed herein. Any isolated, recombinant, or synthetic or chimeric (a combination of synthetic and recombinant) polypeptide (e.g., enzyme or catalytic antibody) can be used, e.g., a chlorophyllase, chlase, or chlorophyll-chlorophyllido hydrolyase or polypeptide having a similar activity can be used in a composition or method as disclosed herein, (e.g., Marchler-Bauer (2007) Nucleic Acids Res. 35:D237-40). Polypeptides and/or peptides having esterase (e.g., chlorophyllase) activity can be used in the compositions or methods as disclosed herein.

For example, in one aspect, a recombinantly produced chlorophyllase protein can be used to enzymatically treat a chlorophyll and/or pheophytin containing biomass (e.g. a product-containing biomass, dry or semi-dry biomass, biomass lysate, hydrophobic extract-1, hydrophobic extract-1 or product (for example, as shown in FIG. 7)). In some aspects, the chlorophyllase can comprise a tag (e.g. biotin, FLAG-tag, or a histidine tag) that allows immobilization or capture of the recombinant enzyme. A chlorophyllase can be prepared from any genetically modified or natural source (e.g. sugar beat leaves or spinach leaves).

Removal of nitrogen containing chlorophyll and/or pheophytin from a biomass can comprise contacting the biomass with one or more chlorophyll degrading enzymes (e.g. chlorophyllase, RCC reductase, a dechelatase, or a pheophorbide a oxygenase). Therefore, in one example, a method for producing a refined product (e.g. nitrogen-depleted product) can comprise degrading a chlorophyll and/or pheophytin in a composition comprising chlorophyll and/or pheophytin and a product. The chlorophyll and/or pheophytin can be degraded by adding an enzyme to the composition. In one example, the enzyme can be a chlorophyllase.

Additional steps of the production process can comprise removing from the composition a cleaved portion of the chlorophyll comprising nitrogen and refining the remainder of the composition to produce a nitrogen-depleted product. The chlorophyll degrading enzymes can be added by any suitable method. For example, the chlorophyll degrading enzymes can be immobilized or fixed to a solid support by any suitable method. The chlorophyll degrading enzymes can be immobilized to any substrate, e.g., filters, fibers, columns, beads, colloids, gels, hydrogels, meshes and the like. The enzyme can be immobilized using any organic or inorganic support. Exemplary inorganic supports include alumina, celite, Dowex-1-chloride, glass beads, and silica gel. Exemplary organic supports include alginate hydrogels or alginate beads or equivalents.

The chlorophyll and/or pheophytin degrading enzymes can be expressed in a fatty acid, lipid and/or hydrocarbon-containing biomass (e.g. a product-containing biomass) by genetic modification of the photosynthetic organisms that comprise the biomass. The expression of a chlorophyll degrading enzyme by a genetically modified photosynthetic organism can be inducible by any suitable regulated promoter system (e.g. a tetracycline inducible promoter system, light inducible promoter, nitrate inducible promoter, or heat responsive promoter).

Enzymes (e.g. chlorophyllases) used in the methods as disclosed herein can be formulated or modified, (e.g., chemically modified), for example to enhance oil solubility, stability, activity, or for immobilization. For example, enzymes used in the methods as disclosed herein can be formulated to be amphipathic or more lipophilic. Enzymes used in the methods as disclosed herein can be encapsulated, (e.g., in liposomes or gels (e.g., alginate hydrogels or alginate beads or equivalents)). Enzymes used in the methods as disclosed herein can be formulated in micellar systems (e.g., a ternary micellar (TMS) or reverse micellar system (RMS) medium). Enzymes used in the methods as disclosed herein can be formulated as described in Yi (2002) J. of Molecular Catalysis B: Enzymatic, Vol. 19-20, pgs 319-325. In one non-limiting example an amphipathic enzyme, (e.g., chlorophyllase), in the form of a ternary micellar (TMS) or reverse micellar system (RMS) medium can be encapsulated in alginate hydrogels. In one aspect, an enzyme (e.g., a chlorophyllase), is prepared in aqueous buffer and retained in a hydrogel, e.g., TMS/alginate and RMS/alginate. One approach to encapsulating an enzyme can be emulsification and/or internal gelation of the enzyme-TMS or -RMS system.

The enzymatic reactions (e.g. chlorophyll hydrolysis reactions) of the methods as disclosed herein can be performed in vitro, and may utilize one reaction vessel or multiple vessels. In one aspect, the enzymatic reactions of the methods as disclosed herein can be performed in a refining apparatus. Enzyme reactions can be mixed or homogenized to increase enzyme activity.

Enzyme reactions (e.g. chlorophyll degrading reactions) can be conducted at temperatures greater than about 25° C. and up to about 95° C., for example. Chlorophyll degrading enzymes (e.g. a chlorophyllase) can retain activity under conditions comprising a temperature of less than about 25° C., however, with significantly reduced activity. Chlorophyll degrading reactions can take place at temperatures greater than about 25° C., about 30° C., about 35° C., about 40° C. and about 45° C. and at temperature less than about 65° C., about 60° C., about 55° C., about 50° C., about 45° C. and about 40° C., for example. In one aspect, the specific activity of a chlorophyll degrading enzyme can be thermostable or thermotolerant at a temperature greater than about 37° C. to about 95° C. A CCP can be treated with a chlorophyll degrading enzyme, for example, at a temperature greater than about 30° C. and less than about 60° C.

Degradation of chlorophyll and/or pheophytin in a biomass by a chlorophyll degrading enzyme can be enhanced by the addition of acetone. Acetone can be added to a chlorophyllase reaction to increase enzyme activity up to a final concentration of about 30% (v/v), for example. In some embodiments, acetone is added to the chlorophyllase reaction up to about 20%. In some embodiments acetone is added to the chlorophyllase reaction up to about 10%.

Degradation of chlorophyll and/or pheophytin in a biomass by a chlorophyll degrading enzyme can also be enhanced by the addition of glycerol. For example, glycerol can be added to a chlorophyllase reaction to increase enzyme activity up to a final concentration of about 5%, about 10%, about 15%, about 20%, about 25%, about 30%, about 35%, about 40%, about 45%, about 50%, about 55%, about 60%, about 65%, about 70%, or about 75% (v/v). In some embodiments, glycerol is added to the chlorophyllase reaction up to about 5%, about 10%, about 20%, or about 30%. In some embodiments, glycerol is added to the chlorophyllase reaction up to about 30%. For example, a method for producing a nitrogen-depleted product can comprise degrading a chlorophyll and/or pheophytin in a composition comprising chlorophyll and/or pheophytin and a product, by hydrolysis. In some aspects, the hydrolysis step comprises adding glycerol to the composition.

Degradation of chlorophyll and/or pheophytin in a biomass by a chlorophyll degrading enzyme can also be enhanced by the addition of divalent cations (e.g. Mg⁺⁺). Addition of a divalent cation (e.g. Mg⁺⁺) up to about 100 mM can significantly increase the activity of a chlorophyll degrading enzyme in a reaction. Higher concentrations of divalent cations, greater than about 100 mM, are also acceptable.

Degradation of chlorophyll and/or pheophytin in a biomass by a chlorophyll degrading enzyme can be enhanced by adjusting the pH of the reaction to, for example, a pH greater than about 6.5 up to a pH of about 12. The pH of the reaction can also be adjusted to up to 7.5, 8.5, 9.5, 10.5, or 11.5.

Degradation of chlorophyll and/or pheophytin in a biomass by a chlorophyll degrading enzyme can be enhanced by the addition of a non-ionic surfactant or detergent (e.g. Triton X-100) up to, for example, about 2% (w/v).

Degradation of chlorophyll and/or pheophytin in a biomass by a chlorophyll degrading enzyme can be enhanced by an incubation or holding period from about 10 minutes up to several days, depending on the amount of enzyme present and the efficiency of the reaction. A highly efficient enzymatic reaction can be complete within minutes. Degradation of chlorophyll in a CCP by a chlorophyll degrading enzyme can require enzyme concentrations, for example, greater than about 1 ug/ml and up to several hundred mgs/ml depending on the purity and specific activity of the enzyme preparation.

Degradation of chlorophyll and/or pheophytin in a biomass by a chlorophyll degrading enzyme can take place in various ratios of buffer and oil. High oil content tends to decrease enzyme activity. Therefore, higher buffer/oil ratios can be used. Degradation of chlorophyll and/or pheophytin in a biomass by a chlorophyll degrading enzyme can proceed, for example, at buffer/oil ratios of greater than about 1/1, about 2/1, about 3/1, about 4/1, about 5/1, about 6/1, about 7/1, about 8/1, about 9/1 and about 10/1 (v/v).

Degradation of chlorophyll (e.g. removal of a phytol side chain from a chlorophyll molecule) or degradation of pheophytin (e.g. removal of a phytol side chain from a pheophytin molecule) in a biomass can also be achieved by a number of other chemical methods. For example, the phytol side chain can be removed under basic hydrolytic conditions such as sodium hydroxide in water, or lithium hydroxide in a water/tetrahydrofuran (THF)/methanol solvent system. Other methods for ester hydrolysis can be used and are described in March's Advanced Organic Chemistry: Reactions, Mechanisms, and Structure, 4th Edition, John Wiley and Sons Inc. (Chapter 10. p 378-386). Degradation of chlorophyll (e.g. removal of a hydrophobic side chain (e.g. phytol) from a chlorophyll molecule) or degradation of pheophytin (e.g. removal of a phytol side chain from a pheophytin molecule) can be accomplished by any means including, but not limited to, any chemical means (e.g. chemical induced hydrolysis, alcoholysis, glycolysis, etc.), enzymatic means (e.g. by use of a chlorophyllase), or physical means (e.g. by extreme temperatures or sonication). A hydrocarbon side chain may also be removed under acidic conditions in aqueous or anhydrous (e.g., less than about 1% water) solutions. Therefore, in one example a method for producing a nitrogen-depleted product can comprise degrading a chlorophyll and/or pheophytin in a composition comprising chlorophyll and/or pheophytin and a product wherein the degrading step comprises hydrolysis, alcoholysis, or glycolysis. The hydrolysis, alcoholysis, or glycolysis process can be achieved by chemical, physical or enzymatic means and may be degraded under acidic conditions or in an anhydrous solution.

Chlorophyll and/or pheophytin can also be removed from a biomass by a non-enzymatic bleaching process. One non-limiting example of a bleaching process includes adsorption of chlorophyll and/or pheophytin to a bleaching clay with subsequent clay disposal. In another example of a bleaching process, chlorophyll and/or pheophytin is removed from the biomass by adsorption to a carbonaceous material (e.g. activated charcoal or similar products). Some non-enzymatic bleaching processes can require the use of a filter and or a filtering step. Therefore, in one example a method of producing a nitrogen-depleted product from a photosynthetic organism comprises removing, without the use of a filter, nitrogen from a composition comprising the photosynthetic organism or parts thereof, and refining the remainder of the composition to produce the nitrogen-depleted product. The depletion of nitrogen in a nitrogen-depleted product can be complete or partial. In some aspects, the adsorbent material is bleaching clay or a carbonaceous material. In one example, removing nitrogen from the composition comprises hydrolyzing chlorophyll and/or pheophytin. In one aspect, removing nitrogen from the composition comprises hydrolyzing chlorophyll and/or pheophytin and further comprises a bleaching clay.

In some aspects, a composition comprising a photosynthetic organism or parts thereof can comprise a genetically modified photosynthetic organism that is lysed (for example crushed or flaked). In some aspects, a photosynthetic organism or parts thereof can comprise live, dead, dried, crushed, flaked, defatted, lysed, or membrane disrupted photosynthetic organisms. In some aspects, a photosynthetic organism or parts thereof is first depleted or partially depleted of a selected protein or carbohydrate. For example, a genetically modified photosynthetic organism can be used to produce a commercially valuable recombinant protein. After removal (e.g. extraction) of the protein from the photosynthetic organism or from the media used to cultivate the photosynthetic organism, the remaining organism can be used to produce a nitrogen-depleted product. In another example, lysis of the photosynthetic organism can be desired to release a chlorophyll hydrolysing enzyme (e.g. a chlorophyllase).

In another aspect, a method of producing a nitrogen-depleted product from a photosynthetic organism comprises removing, without the use of a filter, nitrogen from the composition wherein the method further comprises dissolving chlorophyllide or a pheophorbide in a solvent. In one example, the solvent is water, acetone, glycerol, alcohol, hexane, heptane, methylpentane, toluene, or methylisobutylketone. If the solvent is an alcohol, examples of alchohols include, but are not limited to, methanol, propanol, ethanol, and isopropanol. The method can comprise further refining the nitrogen-depleted product. In some aspects, the removing step comprises use of an enzyme. In one example, the enzyme is chlorophyllase. In one aspect, the nitrogen-depleted product comprises phytol.

In one aspect, is a method of producing a nitrogen-depleted product from a photosynthetic organism wherein the nitrogen-depleted product comprises a fatty acid, lipid, or hydrocarbon. In one example, the nitrogen-depleted product comprises one or more hydrocarbons. In one example, the hydrocarbon is an isoprenoid. In another example, the isoprenoid is a monoterpene, sesquiterpene, diterpene, sesterpene, triterpene, carotenoid, squalene, or neophytadiene. In one example, the method further comprises removing, without the use of a filter, nitrogen from the composition comprising the photosynthetic organism or parts thereof, and refining the remaining composition to produce the nitrogen-depleted product.

In another aspect, a non-enzymatic bleaching step is contemplated to remove chlorophyll when chlorophyll levels are less than about 0.5% of the CCP. A bleaching step can be used following enzymatic degradation of chlorophyll and/or pheophytin in a biomass. A bleaching step can be used following removal of chlorophyll and/or pheophytin degradation products (e.g. chlorophyllide and/or pheophorbide). A bleaching step can be used to remove chlorophyll and/or pheophytin from a biomass. A method of producing a refined product from a composition obtained from a photosynthetic organism can comprise, removing, without the use of a filter, nitrogen atoms from the composition, removing nitrogen (e.g. chlorophyll and/or pheophytin) from the composition by use of a bleaching clay, and refining the composition depleted of nitrogen atoms.

Nitrogen Extraction (i.e. Removal of Chlorophyll Degradation Products)

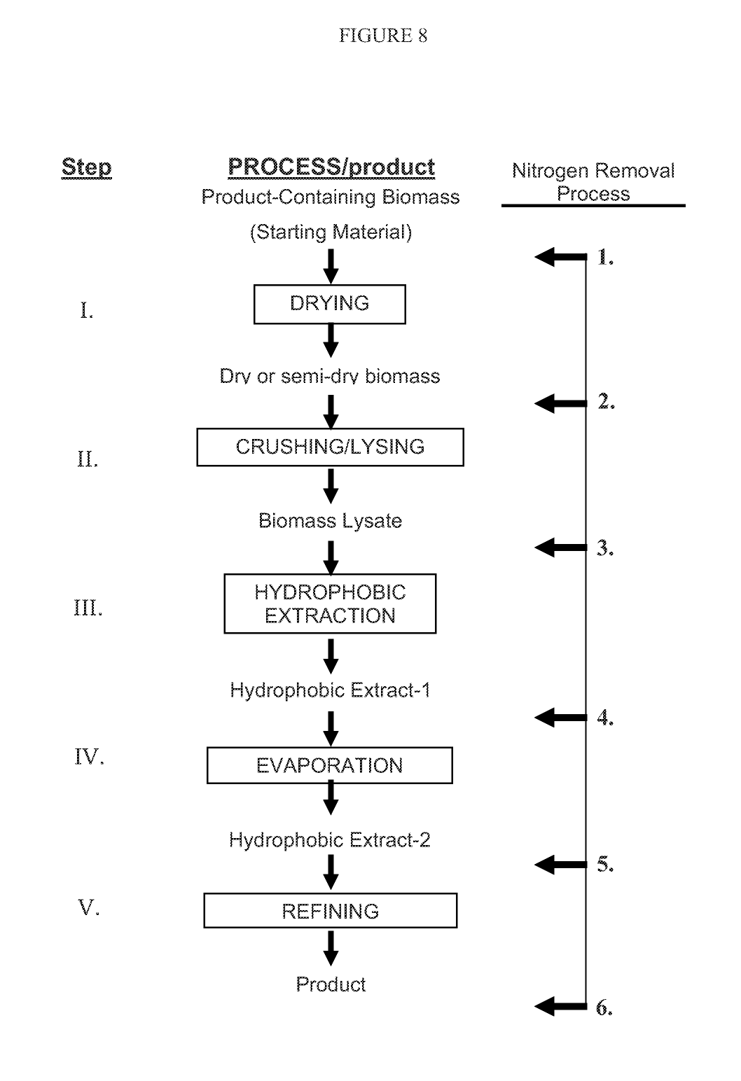

Nitrogen, for example nitrogen contained in a chlorophyll degradation product (e.g. chlorophyllide, pheophorbide, red chlorophyll catabolite, fluorescent chlorophyll catabolite, or any nitrogen containing degradation product of chlorophyll) can be removed from a biomass by integrating an additional nitrogen extraction step in the production process (e.g. see FIG. 8). For example, the nitrogen extraction step can comprise a washing or extraction step using any suitable aqueous or polar solvent, any solvent miscible with water, or any solvent immiscible with the biomass. Nitrogen containing chlorophyll and/or pheophytin degradation products can be retained in the solvent phase. Degradation products that do not contain nitrogen (e.g. phytol) can be retained in the lipid phase of the extraction along with the product of interest (e.g. wherein the product of interest is a biofuel product). Therefore, in one example, a method for producing a nitrogen-depleted product can comprise degrading a chlorophyll and/or pheophytin in a composition comprising chlorophyll and/or pheophytin and a product, removing from the composition a cleaved portion of the chlorophyll and/or pheophytin comprising nitrogen and refining the remaining composition to produce a nitrogen-depleted product. In one aspect, the composition comprises pigments from a photosynthetic organism. In another aspect, the cleaved portion of the chlorophyll that is removed is a chlorophyllide. In one example, the cleaved portion that is removed is a pheophorbide. The nitrogen-depleted product can be a biofuel. The nitrogen-depleted product can comprise a phytol. In another example, a second cleaved portion of the chlorophyll and/or pheophytin is also removed from the composition. In one example, the second cleaved portion is phytol. Additional steps of the production process can comprise further refining the nitrogen-depleted product.

Non-limiting examples of solvents and acids that can be used for nitrogen extraction include water, organic acids, or inorganic acid such as, e.g., acetic acid, formic acid, citric acid, phosphoric acid, succinic acid, nitric acid, sulfuric acid, acetone, alcohols, glycerol, hexane, heptane, methylpentane, toluene, or methylisobutylketone, or any mixtures or salt solutions thereof. In one example, a method for producing a nitrogen-depleted product can comprise removing a cleaved portion of a chlorophyll and/or pheophytin comprising nitrogen from a composition, wherein the method of removing comprises mixing a solvent with the composition and removing the solvent. In one aspect, the solvent can be water, acetone, glycerol, alcohol, hexane, heptane, methylpentane, toluene, or methylisobutylketone. If the solvent is an alcohol, examples of alchohols include, but are not limited to, methanol, propanol, ethanol, and isopropanol. In one aspect, the cleaved portion of the chlorophyll and/or pheophytin comprising nitrogen is retained in the solvent and removed. In one aspect, the cleaved portion of the chlorophyll comprising nitrogen that is retained in the solvent and removed, can be chlorophyllide. The method can further comprise refining the remaining composition to produce a nitrogen-depleted product. In one aspect, the remaining composition comprises the nitrogen-depleted product. The method can further comprise refining the remaining composition (e.g. the nitrogen-depleted product). The refining step can comprise cracking.

In one example, a method for producing a nitrogen-depleted product can comprise removing a cleaved portion of a chlorophyll and/or pheophytin comprising nitrogen from a composition, wherein the method of removing comprises mixing a solvent with the composition and retaining the solvent. In one aspect, the solvent can be hexane. In one aspect, the solvent can be any solvent contemplated for the hydrophobic extraction (for example, as shown in FIG. 7). In one aspect, the cleaved portion of the chlorophyll and/or pheophytin comprising nitrogen is not retained in the solvent and the solvent comprises the product (e.g. the nitrogen-depleted product). In one aspect, the cleaved portion of the chlorophyll comprising nitrogen that is not retained in the solvent is therefore removed from the composition comprising the product. In one aspect, the cleaved portion of the chlorophyll comprising nitrogen can be chlorophyllide. In another aspect, the cleaved portion of the pheophytin comprising nitrogen can be pheophorbide. The method can further comprise, refining the product to produce a nitrogen-depleted product. The method can further comprise refining the remaining composition (e.g. the nitrogen-depleted product). The refining step can comprise cracking.

The nitrogen extraction step can utilize a suitable method of washing or extracting. In one aspect, the nitrogen containing chlorophyll and/or pheophytin degradation products (e.g. chlorophyllide, pheophorbide, red chlorophyll catabolite, fluorescent chlorophyll catabolite, or any nitrogen containing degradation product of chlorophyll) can be partially or completely transferred into the aqueous phase and removed from the lipid phase by two or more washing or extracting steps with a suitable solvent. Extractors, if used, can be operated in crosscurrent or counter-current mode.

The nitrogen extraction step (e.g. by washing or extracting) can be enhanced by modifying the pH (e.g., increasing pH) of the biomass. The pH of a biomass can be modified before or during the nitrogen extraction step. Thus, the compositions, methods and production steps as disclosed herein can also comprise a caustic neutralization step. In one example, the compositions and methods as disclosed herein comprise a neutralization step (e.g. adjusting the pH to greater than about 6). The compositions and methods as disclosed herein can comprise modifying pH to promote aqueous separation of a chlorophyllide and/or pheophorbide.

The methods of removing nitrogen from a biomass (e.g. by a chlorophyll and/or pheophytin degradation step and/or a nitrogen extraction step) can be used before, during, or after any steps or all steps of a production process (e.g. FIG. 8). For example, a collected source material (e.g., algal biomass) is dried and a biomass is extracted using a solvent (e.g., hexane). A bleaching clay may then be used on the miscella (hexane solvent plus extracted lipids). During this process, phytadiene and phytol are cleaved from chlorophyll and pheophytin. Pheophorbide and/or chlorophyllide are adsorbed onto the bleaching clay, which can then be removed (e.g., by filtration). Typically, the solvent (e.g., hexane) is evaporated and the neophytadiene and/or phytol are recovered with the nitrogen-depleted lipid oil product. In one example, the method of removing nitrogen from a biomass by the addition of a chlorophyll degrading enzyme can comprise the addition of the enzyme to the hydrophobic extract-2 (for example, as shown in FIG. 7). In another example, the chlorophyll and/or pheophytin degrading enzyme can be added to the biomass lysate or hydrophobic extract-1. The nitrogen extraction step can take place anytime during or after the chlorophyll and/or pheophytin degradation step. For example, the chlorophyll and/or pheophytin degrading step can take place during the crushing/lysis step and the nitrogen extraction step can take place on the product (i.e. after refining, for example, as shown in FIG. 8). In some aspects, a nitrogen extraction step may not be needed. For example, wherein a biofuel is refined and the chlorophyll and/or pheophytin degradation step precedes a hydrophobic extraction step (for example, as shown in FIG. 7). In this example, a separate nitrogen extraction step may not be needed. In some aspects, the chlorophyll and/or pheophytin degradation and nitrogen extraction steps follow an evaporation step (for example, as shown in FIG. 7, Step 1V). In some aspects the chlorophyll and/or pheophytin degradation and nitrogen extraction steps follow a hydrophobic extraction step (FIG. 7, Step III).

A chlorophyll containing product comprising lipids, fatty acids and/or hydrocarbons obtained from a non-vascular photosynthetic organism may contain, for example, at least 0.1%, 0.2%, 0.3%, 0.4%, 0.5%, 0.6%, 0.7%, 0.08%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% chlorophyll (w/w). For example, a method for producing a nitrogen-depleted product can comprise degrading a chlorophyll in a composition comprising chlorophyll and a product, and removing from the composition a cleaved portion of the chlorophyll comprising nitrogen wherein prior to the degrading step, the composition comprises at least 5% chlorophyll (w/w). In one example, the degrading step can be a hydrolyzing step. Following the production steps disclosed herein (i.e. chlorophyll degradation and nitrogen extraction) the recovered composition can comprise, for example, up to 5%, 4%, 3%, 2%, 1% or 0.5% chlorophyll (w/w). For example, a method for producing a nitrogen-depleted product can comprise degrading a chlorophyll in a composition comprising chlorophyll and a product, and removing from the composition a cleaved portion of the chlorophyll comprising nitrogen, wherein the remaining composition or nitrogen-depleted product comprises less than 1% (w/w) chlorophyll.

In one aspect, is a composition comprising phytol and up to about 0.5% (w/w) chlorophyll or chlorophyllide. In one example, the volume of the composition is greater than 500 liters. In another example, the phytol is at least about 1% of said composition. In another example, the composition further comprises one or more hydrocarbons. The hydrocarbon can be an isoprenoid. The isoprenoid can be a monoterpene, sesquiterpene, diterpene, sesterpene, triterpene, carotenoid, squalene or neophytadiene. In yet another example, the composition can further comprise pigments from a photosynthetic organism. The pigments can be derived from algae.

While certain embodiments have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are intended to provide illustrations of the application of the present invention. The following examples are not intended to completely define or otherwise limit the scope of the invention.

### Example 1

### Nuclear Transformation of C. reinhardtii with a Nucleic Acid Encoding a Chlorophyllase

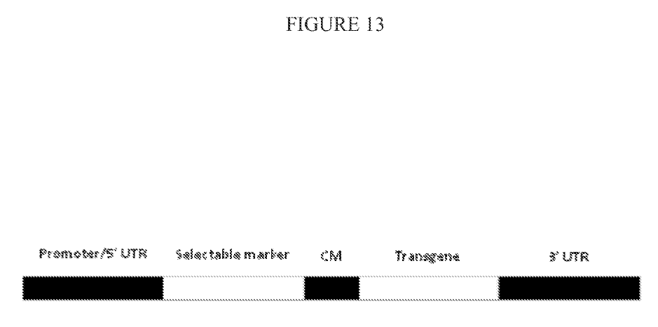

This example describes a method by which a nucleic acid encoding a chlorophyllase from Triticum aestivum (SEQ ID NO.: 27), codon optimized for expression in the C. reinhardtii chloroplast, can be expressed in a green alga. Transforming DNA is shown graphically in FIG. 13. The segment labeled “Transgene” is the T. aestivum chlorophyllase encoding gene, codon optimized for expression in the nuclear genome of C. reinhardtii. The segment labeled “Promoter/5′ UTR” is the C. reinhardtii HSP70/rbcS2 5′ untranslated region with introns, the segment labeled “Selectable Marker” is a bleomycin resistance gene, the segment labeled “CM” (cleavage moiety) is the 2A viral sequence of foot and mouth disease virus (FMDV), and the segment labeled “3′ UTR” is the 3′ untranslated gene region from C. reinhardtii gene rbcS2. The bleomycin resistance gene, 2A and chlorophyllase coding regions can be physically linked in-frame, resulting in a chimeric single ORF. All DNA manipulations can be carried out in the construction of this transforming DNA essentially as described by Sambrook et al., Molecular Cloning: A Laboratory Manual (Cold Spring Harbor Laboratory Press 1989) and Cohen et al., Meth. Enzymol. 297, 192-208, 1998.

For these experiments, transformations can be carried out on C. reinhardtii strain 21gr. Cells can be grown to mid-log phase (approximately 2-6×10⁶ cells/ml) and transformed via electroporation. Tween-20 can be added into cell cultures to a concentration of 0.05% before harvest to prevent cells from sticking to centrifugation tubes. Cells can be centrifuged gently (between 2000 and 5000×g) for 5 min. The supernatant is removed and cells resuspended in TAP+40 mM sucrose media. 1 to 2 ug of transforming DNA is mixed with ˜1×10⁸ cells on ice and transferred to electroporation cuvettes. Electroporation is performed with the capacitance set at 25 uF, the voltage at 800 V to deliver V/cm of 2000 and a time constant for 10-14 ms. Following electroporation, the cuvette can be returned to room temperature for 5-20 min. Cells are transferred to 10 ml of TAP+40 mM sucrose and allowed to recover at room temperature for 12-16 hours with continuous shaking. Cells are then harvested by centrifugation at between 2000 g and 5000 g and resuspended in 0.5 ml TAP+40 mM sucrose medium. 0.25 ml of cells are plated on TAP+20 ug/ml bleomycin. All transformations are carried out under bleomycin selection (20 μg/ml) in which resistance is conferred by the gene encoded by the segment in FIG. 13 labeled “Selection Marker.” Transformed strains are maintained in the presence of bleomycin to prevent loss of the exogenous DNA.

Cells can be screened for expression of the transgenic chlorophyllase by the following method. Patches of algae cells growing on TAP agar plates can be lysed by resuspending cells in 50 μl of 1×SDS sample buffer with reducing agent (BioRad). Samples are then boiled and run on a 10% Bis-tris polyacrylamide gel (BioRad) and transferred to PVDF membranes using a Trans-blot semi-dry blotter (BioRad) according to manufacturer's instructions. Membranes then can be blocked by Starting Block (TBS) blocking buffer (Thermo Scientific) and probed for one hour with mouse anti-FLAG antibody-horseradish peroxidase conjugate (Sigma) diluted 1:3000 in Starting Block buffer. After probing, membranes are washed four times with TBST, then can be developed with Supersignal West Dura chemiluminescent substrate (Thermo Scientific) and imaged using a CCD camera (Alpha Innotech). If cells express the chlorophyllase product, a band at the appropriate molecular weight will be observed in the western blot.

### Example 2

### Transformation and Expression of a gene into C. reinhardtii

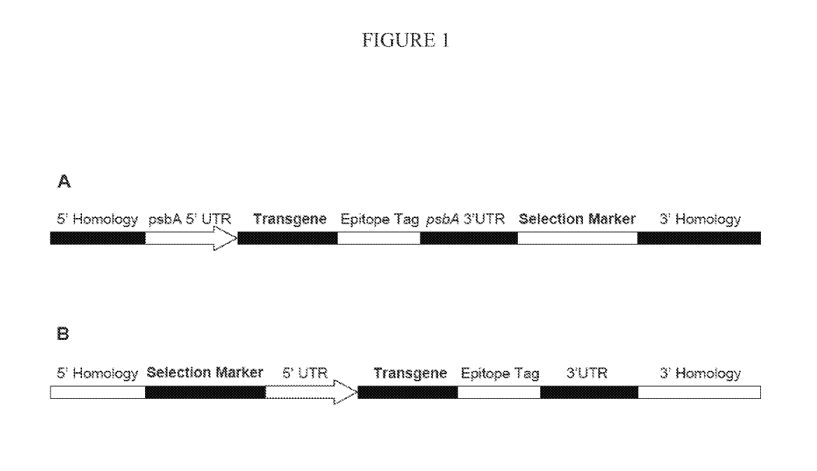

In this example a nucleic acid encoding endo-β-glucanase from T. reesei was introduced into C. reinhardtii. Transforming DNA (SEQ ID NO: 15), codon optimized for expression in the C. reinhardtii chloroplast, is shown graphically in FIG. 1A. In this instance the segment labeled “Transgene” is the endo-β-glucanase protein (SEQ ID NO. 16), the segment labeled “psbA 5′ UTR” is the 5′ UTR and promoter sequence for the psbA gene from C. reinhardtii, the segment labeled “psbA 3′ UTR” contains the 3′ UTR for the psbA gene from C. reinhardtii, and the segment labeled “Selection Marker” is the kanamycin resistance encoding gene from bacteria, which is regulated by the 5′ UTR and promoter sequence for the atpA gene from C. reinhardtii and the 3′ UTR sequence for the rbcL gene from C. reinhardtii. The transgene cassette is targeted to the psbA loci of C. reinhardtii via the segments labeled “5′ Homology” and “3′ Homology,” which are identical to sequences of DNA flanking the psbA locus on the 5′ and 3′ sides, respectively. FIG. 1B is a graphic representation of another exemplary embodiment. All DNA manipulations carried out in the construction of this transforming DNA were essentially as described by Sambrook, Fritsch, Maniatis, Molecular Cloning, A Laboratory Manual, 2nd edition, vol. 1, 2 & 3 Cold Spring Harbor Press, 1989, New York and Cohen et al., Meth. Enzymol. 297, 192-208, 1998.

For these experiments, all transformations were carried out on C. reinhardtii strain 137c (mt+). Cells were grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells were harvested by centrifugation at 4,000×g at 23° C. for 5 min. The supernatant was decanted and cells resuspended in 4 ml TAP medium for subsequent chloroplast transformation by particle bombardment (Cohen et al., Meth. Enzymol. 297, 192-208, 1998). All transformations were carried out under kanamycin selection (150 μg/ml) in which resistance was conferred by the gene encoded by the segment in FIG. 1 labeled “Selection Marker”.

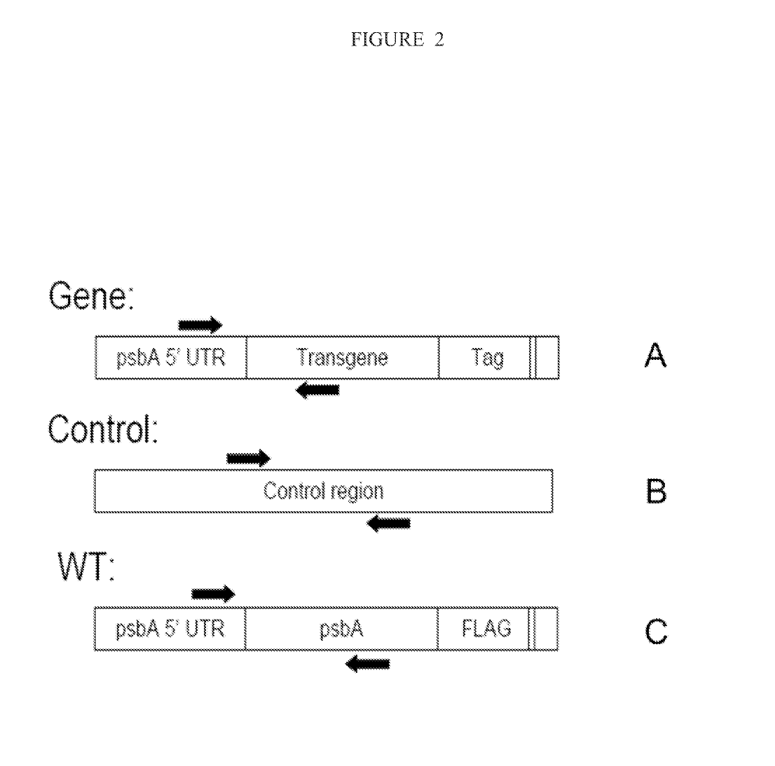

PCR was used to identify transformed strains. For PCR analysis, 10⁶ algae cells (from agar plate or liquid culture) were suspended in 10 mM EDTA and heated to 95° C. for 10 minutes, then cooled to near 23° C. A PCR cocktail consisting of reaction buffer, MgC12, dNTPs, PCR primer pair(s) (Table 1 and shown graphically in FIG. 2A), DNA polymerase, and water was prepared. Algae lysate in EDTA was added to provide template for reaction. Magnesium concentration is varied to compensate for amount and concentration of algae lysate in EDTA added. Annealing temperature gradients were employed to determine optimal annealing temperature for specific primer pairs.

To identify strains that contain the endo-β-glucanase gene, a primer pair was used in which one primer anneals to a site within the psbA 5′UTR (SEQ ID NO. 1) and the other primer anneals within the endo-β-glucanase coding segment (SEQ ID NO. 3). Desired clones are those that yield a PCR product of expected size. To determine the degree to which the endogenous gene locus is displaced (heteroplasmic vs. homoplasmic), a PCR reaction consisting of two sets of primer pairs were employed (in the same reaction). The first pair of primers amplifies the endogenous locus targeted by the expression vector and consists of a primer that anneals within the psbA 5′UTR (SEQ ID NO. 8) and one that anneals within the psbA coding region (SEQ ID NO. 9). The second pair of primers (SEQ ID NOs. 6 and 7) amplifies a constant, or control region that is not targeted by the expression vector, so should produce a product of expected size in all cases. This reaction confirms that the absence of a PCR product from the endogenous locus did not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used was >30 to increase sensitivity. The most desired clones are those that yield a product for the constant region but not for the endogenous gene locus. Desired clones are also those that give weak-intensity endogenous locus products relative to the control reaction.

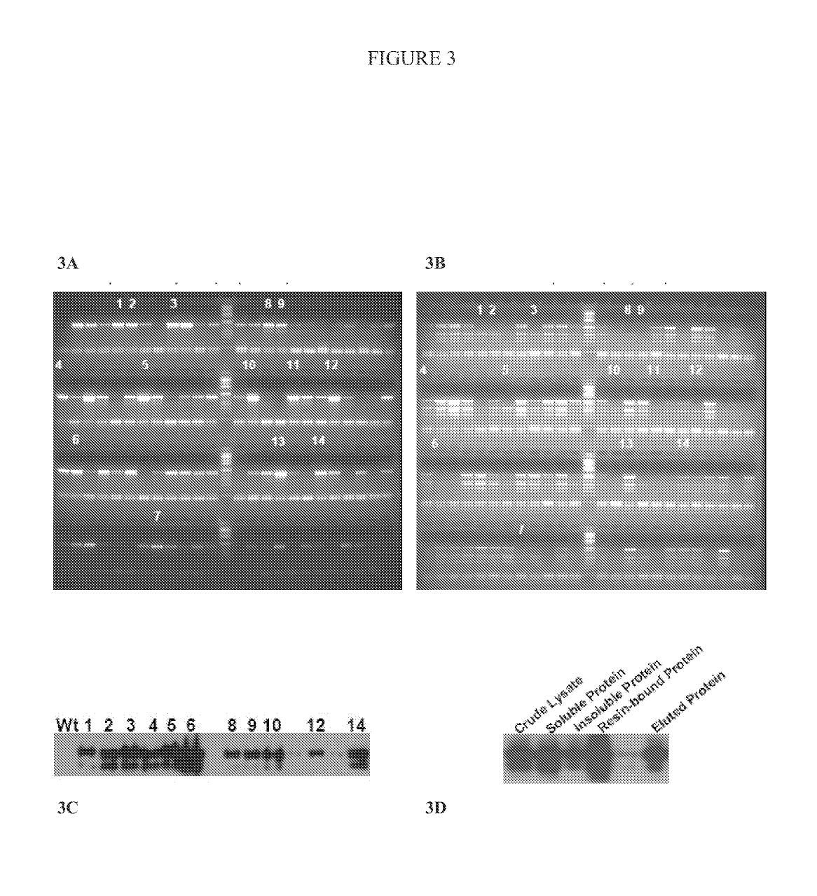

Results from this PCR on 96 clones were determined and the results are shown in FIG. 3. FIG. 3A shows PCR results using the transgene-specific primer pair. As can be seen, multiple transformed clones are positive for insertion of the exo-β-glucanase gene (e.g. numbers 1-14). FIG. 3B shows the PCR results using the primer pairs to differentiate homoplasmic from heteroplasmic clones. As can be seen, multiple transformed clones are either homoplasmic or heteroplasmic to a degree in favor of incorporation of the transgene (e.g. numbers 1-14). Unnumbered clones demonstrate the presence of wild-type psbA and, thus, were not selected for further analysis.

TABLE 1
PCR primers.

| SEQ | | |
|---|---|---|
| ID | | |
| NO. | Use | Sequence |
| 1. | psbA 5′ UTR forward primer | GTGCTAGGTAACTAACGTTTGATTTTT |
| 2. | Exo-β-glucanase reverse primer | AACCTTCCACGTTAGCTTGA |
| 3. | Endo-β-glucanase reverse primer | GCATTAGTTGGACCACCTTG |
| 4. | β-glucosidase reverse primer | ATCACCTGAAGCAGGTTTGA |
| 5. | Endoxylanase reverse primer | GCACTACCTGATGAAAAATAACC |
| 6. | Control forward primer | CCGAACTGAGGTTGGGTTTA |
| 7. | Control reverse primer | GGGGGAGCGAATAGGATTAG |
| 8. | psbA 5′ UTR forward primer | GGAAGGGGACGTAGGTACATAAA |
| | (wild-type) | |
| 9. | psbA 3′ reverse primer | TTAGAACGTGTTTTGTTCCCAAT |
| | (wild-type) | |
| 10. | psbC 5′ UTR forward primer | TGGTACAAGAGGATTTTTGTTGTT |
| 11. | psbD 5′ UTR forward primer | AAATTTAACGTAACGATGAGTTG |
| 12. | atpA 5′ UTR forward primer | CCCCTTACGGGCAAGTAAAC |
| 13. | 3HB forward primer (wild-type) | CTCGCCTATCGGCTAACAAG |
| 14. | 3HB forward primer (wild-type) | CACAAGAAGCAACCCCTTGA |

To ensure that the presence of the endo-β-glucanase-encoding gene led to expression of the endo-β-glucanase protein, a Western blot was performed. Approximately 1×10⁸ algae cells were collected from TAP agar medium and suspended in 0.5 ml of lysis buffer (750 mM Tris, pH=8.0, 15% sucrose, 100 mM beta-mercaptoethanol). Cells were lysed by sonication (5×30 sec at 15% power). Lysate was mixed 1:1 with loading buffer (5% SDS, 5% beta-mercaptoethanol, 30% sucrose, bromophenol blue), and proteins were separated by SDS-PAGE, followed by transfer to PVDF membrane. The membrane was blocked with TBST+5% dried, nonfat milk at 23° C. for 30 min, incubated with anti-FLAG antibody (diluted 1:1,000 in TBST+5% dried, nonfat milk) at 4° C. for 10 hours, washed three times with TBST, incubated with horseradish-linked anti-mouse antibody (diluted 1:10,000 in TBST+5% dried, nonfat milk) at 23° C. for 1 hour, and washed three times with TBST. Proteins were visualized with chemiluminescent detection. Results from multiple clones (FIG. 3C) show that expression of the endo-β-glucanase gene in C. reinhardtii cells resulted in production of the protein.

Cultivation of C. reinhardtii transformants for expression of endo-β-glucanase was carried out in liquid TAP medium at 23° C. under constant illumination of 5,000 Lux on a rotary shaker set at 100 rpm, unless stated otherwise. Cultures were maintained at a density of 1×10⁷ cells per ml for at least 48 hr prior to harvest.

To determine if the endo-β-glucanase produced by transformed alga cells was functional, endo-β-glucanase activity was tested using a filter paper assay (Xiao et al., Biotech. Bioengineer. 88, 832-37, 2004). Briefly, 500 ml of algae cell culture was harvested by centrifugation at 4000×g at 4° C. for 15 min. The supernatant was decanted and the cells resuspended in 10 ml of lysis buffer (100 mM Tris-HCl, pH=8.0, 300 mM NaCl, 2% Tween-20). Cells were lysed by sonication (10×30 sec at 35% power). Lysate was clarified by centrifugation at 14,000×g at 4° C. for 1 hour. The supernatant was removed and incubated with anti-FLAG antibody-conjugated agarose resin at 4° C. for 10 hours. Resin was separated from the lysate by gravity filtration and washed 3× with wash buffer (100 mM Tris-HCl, pH=8.0, 300 mM NaCl, 2% Tween-20). Endo-β-glucanase was eluted by incubation of the resin with elution buffer (TBS, 250 ug/ml FLAG peptide). Results from Western blot analysis of samples collect after each step (FIG. 3D) show that the endo-β-glucanase protein was isolated. A 20 μl aliquot of diluted enzyme was added into wells containing 40 μl of 50 mM NaAc buffer and a filter paper disk. After 60 minutes incubation at 50° C., 120 μl of DNS was added to each reaction and incubated at 95° C. for 5 minutes. Finally, a 36 μl aliquot of each sample was transferred to the wells of a flat-bottom plate containing 160 μl water. The absorbance at 540 nm was measured. The results for two transformed strains indicated that the isolated enzyme was functional (absorbance of 0.33 and 0.28).

### Example 3

### Production of FPP Synthases and Sesquiterpene Synthases in C. reinhardtii

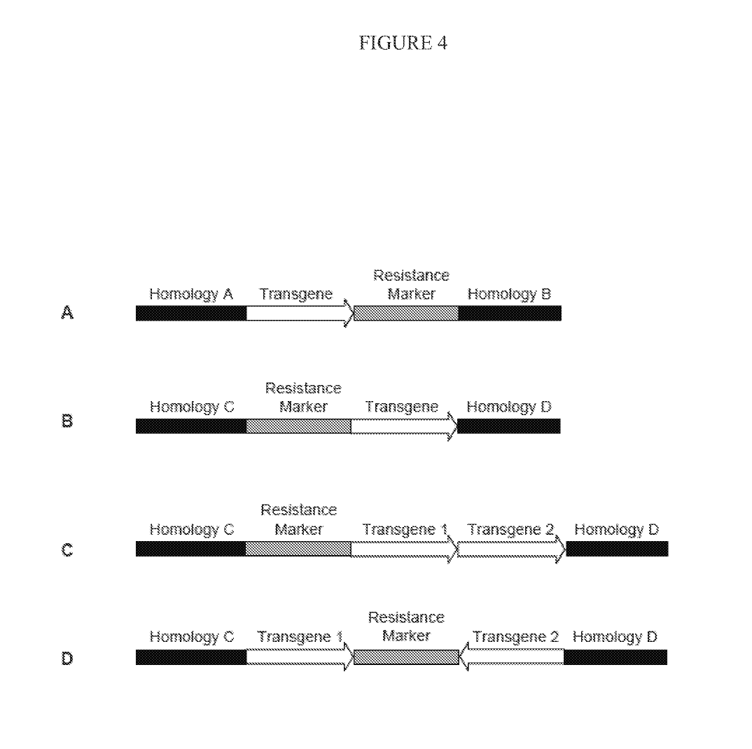

In this example, nucleic acids encoding FPP synthase from G. gallus and bisabolene synthase from P. abies were introduced into C. reinhardtii. Transforming DNA is shown graphically in FIG. 4. In this instance the nucleic acid sequence encoding the FPP synthase gene, codon optimized for expression in the C. reinhardtii chloroplast (SEQ ID NO. 17), is the segment labeled “transgene” in FIG. 4A. The transgene is regulated by the 5′ UTR and promoter sequence for the psbA gene from C. reinhardtii and the 3′ UTR for the psbA gene from C. reinhardtii, and the segment labeled “Resistance Marker” is the kanamycin resistance encoding gene from bacteria, which is regulated by the 5′ UTR and promoter sequence for the atpA gene from C. reinhardtii and the 3′ UTR sequence for the rbcL gene from C. reinhardtii.

The nucleic acid sequence encoding the bisabolene synthase gene, codon optimized for expression in the C. reinhardtii chloroplast (SEQ ID NO. 18; SEQ ID NO: 19) is the segment labeled “transgene” in FIG. 4B and is regulated by the 5′ UTR and promoter sequence for the psbA gene from C. reinhardtii and the 3′ UTR for the psbA gene from C. reinhardtii. The segment labeled “Resistance Marker” is the streptomycin resistance encoding gene from bacteria, which is regulated by the 5′ UTR and promoter sequence for the atpA gene from C. reinhardtii and the 3′ UTR sequence for the rbL gene from C. reinhardtii. The FPP synthase transgene cassette is targeted to the psbA loci of C. reinhardtii via the segments labeled “Homology A” and “Homology B” (FIG. 4A), which are identical to sequences of DNA flanking the psbA loci on the 5′ and 3′ sides, respectively. The bisabolene synthase transgene cassette is targeted to the 3HB locus of C. reinhardtii via the segments labeled “Homology C” and “Homology D” (FIG. 4B), which are identical to sequences of DNA flanking the 3HB locus on the 5′ and 3′ sides, respectively. All DNA manipulations carried out in the construction of this transforming DNA were essentially as described by Sambrook, Fritsch, Maniatis, Molecular Cloning, A Laboratory Manual, 2nd edition, vol. 1, 2 & 3 Cold Spring Harbor Press, 1989, New York and Cohen et al., Meth. Enzymol. 297, 192-208, 1998.

When simultaneous expression of two transgenes is required, DNA can be constructed as described in FIGS. 4C and 4D. The transgene cassette, including promoter, 5′ UTR, gene of interest, and 3′ UTR can be removed from the constructs described in 4A or 4B and combined into a multi-gene expression vector. If constructed with the two transgene cassettes directly adjacent to each other, the resulting DNA will be as shown in FIG. 4C. If constructed with the two transgene cassettes placed on either side of the resistance marker, the resulting DNA will be as shown in FIG. 4D. In either case, transformation of the algae with the DNA will place two separate transgenes into the 3HB locus of C. reinhardtii via the segments labeled “Homology C” and “Homology D” (FIG. 4C or 4D), and will result in an algal strain expressing two separate transgene products.

For these experiments, all transformations were carried out on C. reinhardtii strain 137c (mt+). Cells were grown to late log phase (approximately 7 days) in the presence of 0.5 mM 5-fluorodeoxyuridine in TAP medium (Gorman and Levine, Proc. Natl. Acad. Sci., USA 54:1665-1669, 1965, which is incorporated herein by reference) at 23° C. under constant illumination of 450 Lux on a rotary shaker set at 100 rpm. Fifty ml of cells were harvested by centrifugation at 4,000×g at 23° C. for 5 min. The supernatant was decanted and cells resuspended in 4 ml TAP medium for subsequent chloroplast transformation by particle bombardment (Cohen et al., Meth. Enzymol. 297, 192-208, 1998). Transformations carried out with DNA containing the kanamycin resistance marker were selected under kanamycin at 100 μg/ml; transformations carried out with DNA containing the streptomycin resistance marker were selected under streptomycin at a concentration of 50 μg/ml.

PCR was used to identify transformed strains. For PCR analysis, 10⁶ algae cells (from agar plate or liquid culture) were suspended in 10 mM EDTA and heated to 95° C. for 10 minutes, then cooled to near 23° C. A PCR cocktail consisting of reaction buffer, MgCl2, dNTPs, PCR primer pair(s) (Table 2), DNA polymerase, and water was prepared. Algae lysate in EDTA was added to provide a template for the reaction. Magnesium concentration is varied to compensate for amount and concentration of algae lysate in EDTA added. Annealing temperature gradients were employed to determine the optimal annealing temperature for the specific primer pairs.

To identify strains that contain the FPP synthase gene, a primer pair was used in which one primer anneals to a site within the psbA 5′UTR (SEQ ID NO. 20) and the other primer (SEQ ID NO. 21) anneals within the FPP synthase coding segment. Desired clones are those that yield a PCR product of expected size. To identify strains that contain the bisabolene synthase gene, a primer pair was used in which one primer anneals to a site within the psbA 5′UTR (SEQ ID NO. 20) and the other primer anneals within the bisabolene synthase coding segment (SEQ ID NO. 22). Desired clones are those that yield a PCR product of expected size in both reactions.

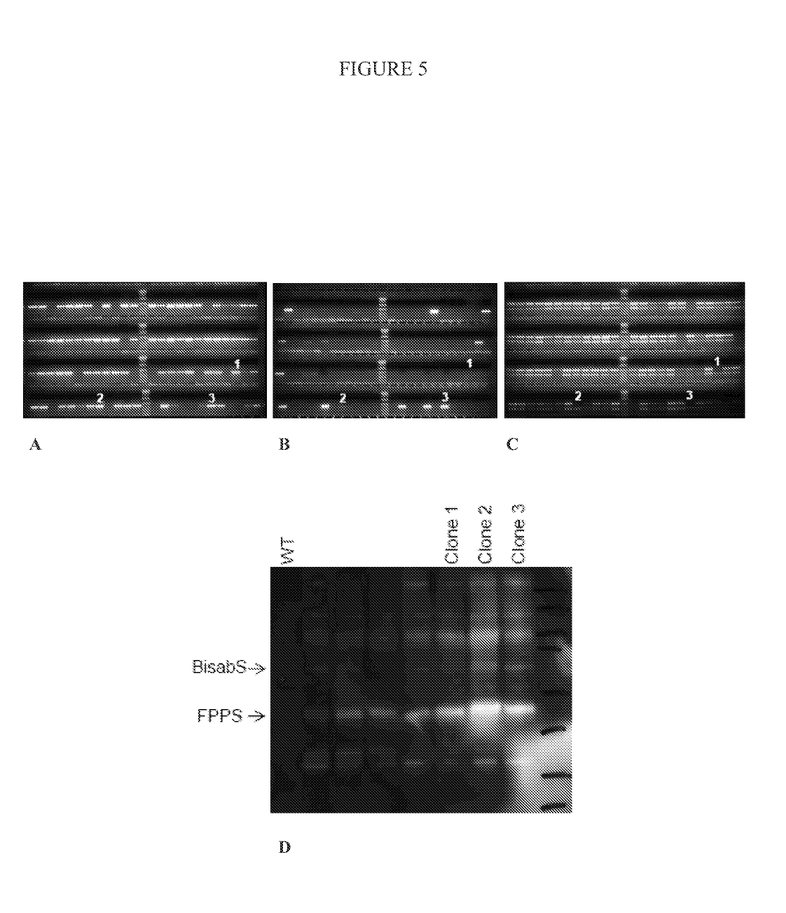

To determine the degree to which the endogenous psbA gene locus is displaced (heteroplasmic vs. homoplasmic), a PCR reaction consisting of two sets of primer pairs were employed (in the same reaction). The first pair of primers amplifies the endogenous locus targeted by the expression vector and consists of a primer that anneals within the psbA 5′UTR (SEQ ID NO. 23) and one that anneals within the psbA coding region (SEQ ID NO. 24). The second pair of primers (SEQ ID NOs. 25 and 26) amplifies a constant, or control region that is not targeted by the expression vector, and should produce a product of expected size in all cases. This reaction confirms that the absence of a PCR product from the endogenous locus did not result from cellular and/or other contaminants that inhibited the PCR reaction. Concentrations of the primer pairs are varied so that both reactions work in the same tube; however, the pair for the endogenous locus is 5× the concentration of the constant pair. The number of cycles used was >30 to increase sensitivity. The most desired clones are those that yield a product for the constant region but not for the endogenous gene locus. Desired clones are also those that give weak-intensity endogenous locus products relative to the control reaction. Results from this PCR are shown in FIG. 5, panels A, B, and C.

To determine if the FPP synthase gene led to expression of the FPP synthase and if the bisabolene synthase gene led to expression of the bisabolene synthase in transformed algae cells, both soluble proteins were immunoprecipitated and visualized by Western blot. Briefly, 500 ml of algae cell culture was harvested by centrifugation at 4000×g at 4° C. for 15 min. The supernatant was decanted and the cells resuspended in 10 ml of lysis buffer (100 mM Tris-HCl, pH=8.0, 300 mM NaCl, 2% Tween-20). Cells were lysed by sonication (10×30 sec at 35% power). Lysate was clarified by centrifugation at 14,000×g at 4° C. for 1 hour. The supernatant was removed and incubated with anti-FLAG antibody-conjugated agarose resin at 4° C. for 10 hours. Resin was separated from the lysate by gravity filtration and washed 3× with wash buffer (100 mM Tris-HCl, pH=8.0, 300 mM NaCl, 2% Tween-20). Resin was mixed 4:1 with loading buffer (XT Sample buffer; Bio-Rad), samples were heated to 95° C. for 1 min, cooled to 23° C., and insoluble proteins were removed by centrifugation. Soluble proteins were separated by SDS-PAGE, followed by transfer to PVDF membrane. The membrane was blocked with TBST+0.5% dried, nonfat milk at 23° C. for 30 min, incubated with anti-FLAG, alkaline phosphatase-conjugate antibody (diluted 1:2,500 in TBST+0.5% dried, nonfat milk) at 4° C. for 10 hours, and washed three times with TBST. Proteins were visualized with chemifluorescent detection. Results from multiple clones (FIG. 5D) show that expression of the FPP synthase gene led to expression of the FPP synthase and expression of the bisabolene synthase gene led to expression of the bisabolene synthase.

Cultivation of C. reinhardtii transformants for expression of FPP synthase and bisabolene synthase was carried out in liquid TAP medium at 23° C. under constant illumination of 5,000 Lux on a rotary shaker set at 100 rpm, unless stated otherwise. Cultures were maintained at a density of 1×10⁷ cells per ml for at least 48 hr prior to harvest.

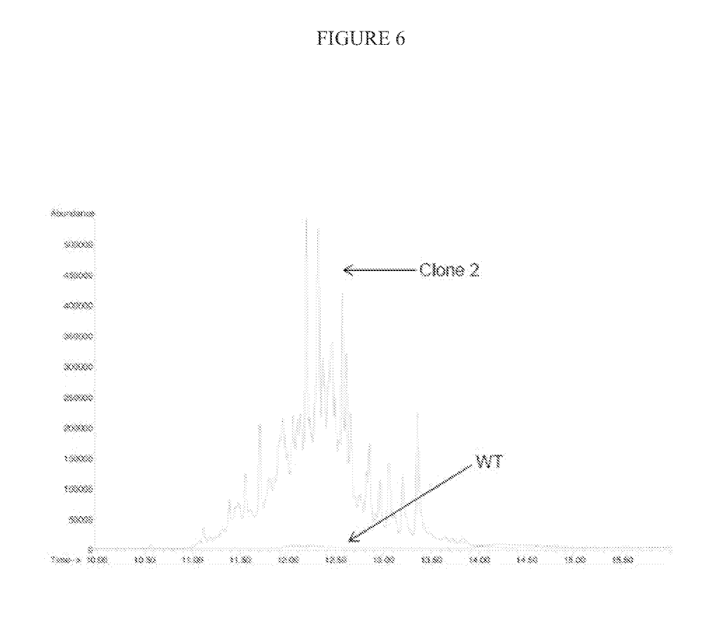

To determine whether bisabolene synthase produced in the algae chloroplast is a functional enzyme, sesquiterpene production from FPP was examined. Briefly, 50 mL of algae cell culture was harvested by centrifugation at 4000×g at 4° C. for 15 min. The supernatant was decanted and the cells resuspended in 0.5 mL of reaction buffer (25 mM HEPES, pH=7.2, 100 mM KCl, 10 mM MnCl₂, 10% glycerol, and 5 mM DTT). Cells were lysed by sonication (10×30 sec at 35% power). 0.33 mg/mL of FPP was added to the lysate and the mixture was transferred to a glass vial. The reaction was overlaid with heptane and incubated at 23° C. for 12 hours. The reaction was quenched and extracted by vortexing the mixture. 0.1 mL of heptane was removed and the sample was analyzed by gas chromatography-mass spectrometry (GC-MS). Results are shown in FIG. 6.

TABLE 2

| SEQ | | |
|---|---|---|
| ID | | |
| NO. | Use | Sequence |
| 20 | psbA 5′ UTR forward primer | GTGCTAGGTAACTAACGTTTGATTTTT |
| 21 | FPP synthase reverse primer (163) | CGTTCTTCTGAGAAATGGCTTA |
| 22 | Bisabolene synthase reverse primer | GACGTTCTTGACGTTTTGTTTG |
| | (307) | |
| 23 | psbA 5′ UTR forward primer | GGAAGGGGACGTAGGTACATAAA |
| | (wild-type) | |
| 24 | psbA 3′ reverse primer | TTAGAACGTGTTTTGTTCCCAAT |
| | (wild-type) | |
| 25 | Control forward primer | CCGAACTGAGGTTGGGTTTA |
| 26 | Control reverse primer | GGGGGAGCGAATAGGATTAG |

### Example 4

### Linoleic Acid Production by Genetically Modified C. reinhardtii and Chlorophyll Hydrolysis and Removal of Chlorophyllide Byproduct

The microalgae Chlamydomonas rheinhardii can be genetically engineered to produce linoleic acid. Production of linoleic acid in C. rheinhardii is achieved by engineering the microalgae to express the heterologous enzyme thioesterase in a chloroplast using the methods described in Examples 1, 2, and 3. The transformed microalgae can be grown in a photobioreactor to a suitable confluency. The microalgae biomass is harvested and dried to about 5% water content, followed by crushing the semi-dry biomass. The semi-dry biomass is extracted with hexane utilizing a solid-liquid extractor system. The aqueous extract is discarded and the lipid extract is retained for further refining. Hexane is removed by evaporation. The resulting product comprises linoleic acid and chlorophyll.

Chlorophyll is removed from the linoleic containing product by the enzyme-catalyzed hydrolysis of chlorophyll. A reaction mixture is prepared in a 50-mL Erlenmeyer flask and consists of 1.1 mL of Tris-HCl buffer solution (20 mM, pH 8.0), 0.3 mL of acetone, and 0.6 mL of linoleic oil product comprising about 20% (w/v) chlorophyll. The enzymatic reaction is initiated by the addition of 1 mL a chlorophyllase suspension, containing 200 ug protein, to the reaction medium. The buffer to oil ratio is about 4 to 1. The mixture is incubated at 45° C. with continuous agitation at 200 rpm using an orbital shaker-incubator (New Brunswick Scientific, Edison, N.J.) for 2 h. The enzymatic reaction is prepared for chlorophyllide extraction by the addition of 5 mL of Tris-NaCl solution (20 mM Tris, 150 mM NaCl, pH 8.0). The reaction is mixed thoroughly and subjected to centrifugation at 1000×g for 30 minutes. The aqueous phase containing the chlorophyllide is removed and the nitrogen-depleted linoleic acid product is recovered. This method can be scaled up as needed.

### Example 5

### Nitrogen Depletion and Phytol Extraction

In this example, phytol and neophytadiene are extracted from genetically modified C. reinhardtii. A C. reinhardtii strain is dried at 80° C. for 3 days. The dried biomass is ground into a powder form. About 2 kg of the powder is extracted with isopropanol at 60° C. with a solvent to algae ratio of about 10 to 1 by mass. The extraction is performed in a rotating 20 L round bottom glass container for a period of about 24 h. The miscella (solvent plus extracted lipids) is decanted and filtered with a 20 um filter. The isopropanol is removed in a rotary evaporator. About 150 g of lipid oil product is obtained. Hexane (at a 10 to 1 ration by weight) is added to 10 g of the lipid oil. Bleaching clay (Oil-dri Perform 6000) is added to hexane solution in the amount of 2:1 by weight of bleaching clay to oil. The solution is mixed for 4 hours at room temperature. The solution is then filtered through 2.5 um filter paper to remove the bleaching clay and adsorbed pheophorbide. The hexane is removed by rotary evaporator. The oil is analyzed on GC/FID. The oil contains 7% neophytadiene and 2% phytol by weight. The chlorophyll concentration in the oil decreases by a certain percentage. The nitrogen content also decreases by a certain percentage.

### Example 6

### Integration of a Nitrogen Removal Process into a Refining Method

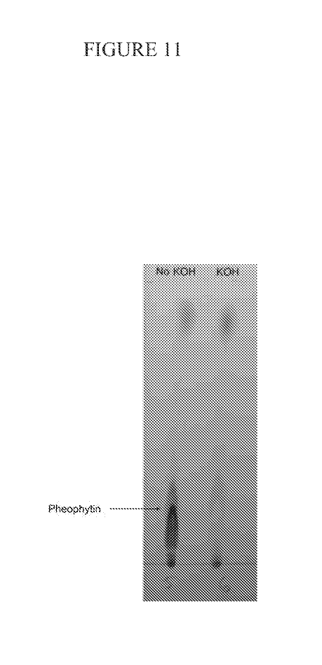

A wet biomass solution containing 40 g (ash free dry weight of algae Tetraselmis) and 600 g of water and media was pretreated with KOH (1 molar) at 60° C. for 1 hour. A second wet biomass solution containing 40 g (ash free dry weight of algae Tetraselmis) and 600 g of water and media was not treated with KOH. Both solutions were then brought to pH 2 with concentrated sulfuric acid. Extraction was done in a bead mill with the solution and 600 g of Heptane at 70° C. for 30 minutes. The emulsion from the bead mill was centrifuged to separate the aqueous phase containing the extracted biomass from the organic phase containing the oil product and heptane. Fresh heptane was added back to the bead mill with the aqueous phase and extracted biomass, for a second extraction, followed by another phase separation step with the centrifuge. This was repeated one more time to give a total of three extractions and phase separations, for each biomass solution. As can be seen in the photograph of a thin layer chromatography (TLC) plate (FIG. 11), chlorophyll in the form of pheophytin was mostly removed from product oil from the KOH pretreated biomass (lane 2 of FIG. 11), but not from biomass without the KOH pretreatment (lane 1 of FIG. 11).

### Example 7

### Base Hydrolysis Results in Higher Levels of Phytol in Oil

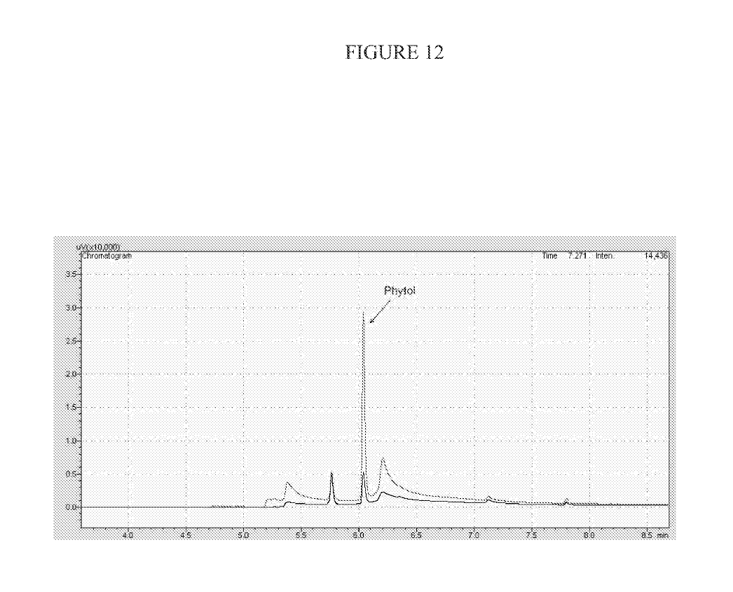

FIG. 12 shows gas chromatograms for the two oils from the extraction described above: one oil sample is from base hydrolysed biomass (lighter line) and the other oil sample is from biomass that was not base hydrolysed (darker line). The chromatogram for the oil from the base hydrolysed biomass (lighter line) shows a much greater peak for phytol than the other oil (darker line). The concentration of phytol in oil from base hydrolysis was 14.2% and the concentration in the oil from biomass that was not base hydrolysed was only 2.2%. This shows that phytol was cleaved and recovered by one embodiment of the method.

### Example 8

### Scale Up of Integration of a Nitrogen Removal Process into a Refining Method

This example describes a commercial scale process with three extraction stages operating in continuous counter-current mode. The extraction solvent is toluene. There is a decanting step after each extraction stage to separate the aqueous phase from the organic phase. The feed to the process is wet biomass with a flow rate of 3,000,000 kg/h. The feed is composed of 10% ash free dry weight algae (AFDW) and 90% water and media. The feed solution is pretreated by adding KOH to bring the pH to 10 in a continuous stirred tank (CSTR) at 60° C. The outlet from the CSTR is fed to extraction tank number #1. The organic phase from extraction stage #2 is the also fed to extraction tank #1 at a flow rate of 2,000,000 kg/h. The mean residence time in the extraction tanks is 20 minutes and the operating temperature is 120° C. The outlet from extraction tank #1 is decanted. The organic phase is sent to an evaporator to remove the toluene solvent and to recover the valuable lipid product. The aqueous phase from the third extraction phase exits the process and is a biproduct stream. It contains the cleaved porphorin ring of chlorophyll containing nitrogen and the residual biomass.

1-227. (canceled)

228. A method for producing a nitrogen-depleted product from an non-vascular photosynthetic organism comprising obtaining a biomass composition from the non-vascular photosynthetic organism, wherein the biomass composition comprises one or more chlorophylls and/or one or more pheophytins and an oil; degrading at least a subset of the chlorophyll or pheophytin in the biomass composition by heating the biomass to 60° C. to 250° C. in the presence of a base and one or more solvents; removing a cleaved portion of the degraded chlorophyll or pheophytin by removing the one or more solvents, wherein the cleaved portion comprises nitrogen; and refining the biomass composition to produce a nitrogen-depleted product.

229. The method of claim 228, wherein the biomass is heated to 80° C. to 200° C.

230. The method of claim 229, wherein the biomass is heated to 120° C.

231. The method of claim 228, wherein the solvent is at least one of water, acetone, glycerol, alcohol, hexane, heptane, methylpentane, toluene and methylisobutylketone.

232. The method of claim 231, wherein the alcohol is at least one of methanol, propanol, ethanol and isopropanol.

233. The method of claim 228, wherein the solvent is an oil immiscible solvent.

234. The method of claim 233, wherein the solvent is water.

235. The method of claim 228 wherein the base is bleach, sodium hydroxide, potassium hydroxide, ammonia, sodium carbonate, calcium carbonate, calcium hydroxide, or a solid base catalyst

236. The method of claim 235, wherein the sold base catalyst is calcium methoxide, calcium oxide, potassium hydroxide/aluminum oxide, or magnesium oxide.

237. The method of claim 228, wherein the degrading occurs at a pH between 6.5 and 12.

238. The method of claim 237, wherein the pH is 7.5, 8.5, 9.5, 10, 10.5 or 11.5

239. The method of claim 238, wherein the pH is 10.

240. The method of claim 228, wherein said method does not involve addition of an adsorbent material.

241. The method of claim 228, wherein the non-vascular photosynthetic organism is a eukaryote.

242. The method of claim 241, wherein the eukaryote is an alga.

243. The method of claim 242, wherein the alga is a green alga.

244. The method of claim 243, wherein the green alga is a Chlorophycean.

245. The method of claim 244, wherein the green the Chlorophycean is a Chlamydomonas, Scenedesmus, Chlorella or Nannochloropsis.

246. The method of claim 228, wherein the non-vascular photosynthetic organism is a prokaryote.

247. The method of claim 246, wherein the prokaryote is a cyanobacterium.
